(19) United States
(12) Patent Application Publication
(10) Pub. No.: US 2010/0015195 A1
(43) Pub. Date: Jan. 21, 2010

(54) INJECTABLE DEPOT COMPOSITIONS AND IT'S PROCESS OF PREPARATION

(76) Inventors: Rajesh Jain, New Delhi (IN); Kour Chand Jindal, New Delhi (IN); Sampath Kumar Devarajan, New Delhi (IN)

Correspondence Address: LADAS & PARRY LLP, 26 WEST 61ST STREET, NEW YORK, NY, 10023, US

(21) Appl. No.: 12/444,197

(22) PCT Filed: Oct. 3, 2007
(86) PCT No.: PCT/IN2007/000454

§ 371 Date: Aug. 31, 2009

(30) Foreign Application Priority Data
Oct. 5, 2006 (IN) 2195/DEL/2006

(51) Int. Cl. A61K 31/496; A61K 31/4196; A61K 31/445; A61K 31/498; A61K 9/00; A61K 9/14; A61P 35/00

(52) U.S. Cl. 424/422; 514/383; 514/319; 514/259.41; 514/254.04; 514/253.07; 424/489; 977/788

(57) ABSTRACT

Novel injectable depot compositions are provided comprising at least one active agent(s) optionally with one or more pharmaceutically acceptable excipient(s) in the form of a multi-component system preferably comprising at least two components which when administered to a subject in need thereof forms an in situ gel depot or implant at the site of injection upon contact with body fluids. Also described are process for preparation of such compositions and method of using such compositions.

### FIELD OF THE INVENTION

The present invention provides novel injectable in situ gelling depot or implant compositions exhibiting minimal burst release comprising at least one active agent(s) or its pharmaceutically acceptable salts, derivatives, isomers, polymorphs, solvates, hydrates, analogues, enantiomers, tautomeric forms or mixtures thereof at least one biodegradable polymer(s), at least one viscosity enhancing agent(s) and optionally one or more pharmaceutically acceptable excipient(s), wherein the compositions are formulated as reconstitutable biodegradable microparticles or nanoparticles, and wherein the said compositions are in the form of a multi-component system preferably comprising at least two components, and wherein the said compositions provide a prolonged release of the active agent(s) for extended periods of time. The present invention also describes process for preparation of such compositions and method of using such compositions. Preferably the compositions increase the efficacy of treatment associated with particularly chronic diseases, leading to greater patient compliance.

### BACKGROUND OF THE INVENTION

It is often desirable to administer drugs using controlled or sustained release formulations that can maintain therapeutic blood levels of the active agent (drug) over extended periods of time. These controlled release formulations reduce the frequency of dosing for enhanced patient convenience and compliance, and also reduce the severity and frequency of side effects. By maintaining substantially constant blood levels and avoiding blood level fluctuations of the drug particularly associated with conventional immediate release formulations that are administered several times a day, controlled or sustained release formulations can provide a better therapeutic profile than is obtainable with conventional immediate release formulations. It is also often desirable to extend the release time of an injected drug to increase its duration of action, or to reduce its toxic effects. Formulations that are readily soluble in the body are usually absorbed rapidly and provide a sudden burst of available drug as opposed to a more desirable and gradual release of the pharmacologically active agent. This ‘burst’ release often results in a substantial portion of the beneficial agent, if not all, being released in a very short time, e.g., hours or 1-2 days. Several attempts have been made to provide controlled release compositions, but have not succeeded in overcoming certain problems associated with long acting parenteral dosage forms, such as achieving an extended release over desired period, stability in tissue fluids, reduced toxicity, reproducibility in preparation, and elimination of undesired physical, biochemical, or toxicological effects associated with the compositions.

Where patient compliance is an issue, a probable approach is to design long acting dosage form compositions of the medication, that is, dosage forms where a single administration leads to a sustained release of the medication over an extended period of time. This, in turn, simplifies the dosage regimen that a patient needs to adhere to, thus reducing the opportunity for non-compliance that occurs with a more rigorous schedule of frequent administration. Among such dosage forms is the depot formulation, which can be administered in various ways including intramuscularly or subcutaneously by injection. The depot injection is specifically formulated to provide a sustained release of the medication over an extended period of time like days, weeks, months or even up to years, as in case of parenteral sustained release formulations.

The use of injectable implants for the delivery of drugs is well known. Both biodegradeable and non-biodegradeable implant versions have been marketed since the 1980s. Examples of these are Zoladex®, a polylactide-co-glycolide formulation of goserelin for the treatment of breast cancer and Norplant®, a non-biodegradeable silicone device for contraception. Small, injectable microparticle formulations are also well known, an example being Lupron Depot®, a formulation of leuprolide for the treatment of prostate cancer. A drawback of such preformed delivery systems is administration. Cylindrical rods such as Zoladex® require relatively large bore needles for implantation. However, injectable formulations comprising microparticles or nanoparticles allow smaller bore needles to be used for in vivo administration. More recently formulations have been developed which are injected as a liquid, but undergo a change to a solid formulation in vivo, which are referred to as ‘in situ gelling systems’. These formulations can be injected intramuscularly or subcutaneously through small bore needles and employ only biocompatible solvents.

Aromatase inhibitors are a class of compounds that act systematically to inhibit oestrogen synthesis in tissues. These compounds prevent oestrogen biosynthesis by inhibiting the enzyme aromatase, which catalyses the conversion of adrenal androgens (androstenedione and testosterone) to oestrogens (oestrogen and oestradiol). There has therefore been interest in developing these compounds as potential therapies for hormone responsive breast cancer in post-menopausal women. Anastrozole (ARIMIDEX®) is a non-steroidal aromatase inhibitor which is highly selective, well tolerated and is effective in treating advanced breast cancer. Donepezil and its salts, have application in the treatment of a variety of disorders, including dementia and attention deficit disorder. In particular, donepezil hydrochloride is employed as a pharmaceutically active agent for the symptomatic treatment of mild to moderate Alzheimer's dementia and is currently formulated as film-coated tablets of 5 mg and 10 mg doses for once a day oral administration under the trade name ARICEPT®.

US Publication No. 20020034532 discloses injectable depot gel composition comprising a biocompatible polymer; a solvent that dissolves the biocompatible polymer and forms a viscous gel; a beneficial agent; and an emulsifying agent in the form of a dispersed droplet phase in the viscous gel. U.S. Pat. No. 6287588 claims a dual phase polymeric agent-delivery composition comprising a continuous biodegradable hydrogel phase, a discontinuous particulate phase comprising defined microparticles; and an agent to be delivered contained in at least said discontinuous particulate phase. The bioactive agent release is described to be modulated by microparticle phase alone or in both the microparticle and the gel matrix. The invention describes a reverse thermal gelation type of matrix. However, the said invention does not describe through clear illustrations the polymeric hydrogel formation at the injection site by non solvent effect by a using an unhydrated cellulosic polymer in the reconstituted suspension composition having easy syringibility to be used as a depot injection.

US Publication No. 20040146562 pertains to a pharmaceutical kit for preparing an injectable depot formulation comprising a solubilized or unsolubilized aryl-heterocyclic compound; and a liquid vehicle comprising a viscosity agent, with the proviso that when said aryl-heterocyclic compound is unsolubilized, said liquid vehicle further contains a solubilizer. US Publication No. 20020034532 discloses injectable depot gel composition comprising a biocompatible polymer, a solvent that dissolves the biocompatible polymer and forms a viscous gel; a beneficial agent; and an emulsifying agent in the form of a dispersed droplet phase in the viscous gel. German Patent No. DE19847593 relates to a composition for parenteral administration comprising an active agent and a carrier material consisting of spherical microparticles of average diameter 1 nm to 100 μm, and at least partly of water-insoluble linear polysaccharide. US Publication No. 20050153841 discloses a formulation for parenteral administration to a subject comprising at least one water miscible solvent; at least one gelling agent; and at least one active agent; characterized in that the gelling agent is in particulate form and suspended in the solvent. However, the said invention does not describe the dual modulation of drug release patterns by means of simultaneously using gelling system dispersed with release controlling particulate form of drug in biodegradable microparticles.

US Publication No. 20060154918 discloses an injectable nanoparticulate olanzapine composition comprising olanzapine nanoparticles having an effective average particle size that results in a therapeutic efficacy of about one week or greater; at least one surface stabilizer; and a pharmaceutically acceptable carrier. US Publication No. 20060193825 discloses pharmaceutical formulations comprising a solid ionic complex of a polypeptide having an isoelectric point lower than physiological pH and an anionic carrier molecule. US Publication No. 20040024069 describes an injectable depot composition comprising a bioerodible, biocompatible polymer; a solvent having a miscibility in water of less than or equal to 7% at 25° C., in an amount effective to plasticize the polymer and form a gel therewith, wherein said solvent is an aromatic alcohol; a thixotropic amount of a thixotopic agent mixed with the polymer solution effective to form a thixotropic composition, the thixotropic agent being selected from the group consisting essentially of lower alkanols and said amount being less than 15 weight percent of the combined weight of the solvent and the thixotropic agent; and a beneficial agent. US Publication No. 2005163859 pertains to a composition comprising a salt comprising a pharmaceutically active compound and a lipophilic counterion; and a pharmaceutically acceptable solvent; wherein the salt and the solvent form a solution and wherein at least a portion of the salt precipitates when the composition is injected into water. US Publication No. 20040138237 describes an injectable depot formulation that is viscous, or becomes viscous in situ, comprising a solubilized ziprasidone. The solubilized ziprasidone cyclodextrin lyophilized complex is suspended in non-aqueous viscosity agents like aluminum monostearate gelled sesame oil; and in situ gelling system such as e.g. stearic acid and N-methyl pyrrolidone. PCT publication no. WO200726145 and WO200726138 describe an in situ gelling and implant formulation comprising anastrozole, a polylactide polymer or poly(lactide-co-glycolide) co-polymer and a solvent.

Investigations in controlled release research has been proceeding especially to obtain a 1-2 month delivery system for biologically active agents or polypeptides using poly(lactide/glycolide) polymers. However, most of these systems have one or more of the following problems: poor encapsulation efficiency and large ‘burst release’ followed by an intermediate ‘no release’ or ‘lag phase’ until the polymer degrades. In general, release from these polymers occurs over a period from about 4 weeks to about several months. In addition, in order to achieve this release substantially high quantities of high molecular weight hydrophobic polymers had been generally used which often results in residual polymer remaining at the site of administration long after the release of active core. The present invention provides novel in situ gelling depot or implant compositions which alleviates the limitations of the prior art.

Several attempts to provide dosage form compositions to sustain medication levels including the use of biodegradable materials for delivery of active agent for extended periods of time have been described previously. Many sustained release parenteral compositions described in the prior art can exhibit an increased release of biologically active agent over the first twenty-four hours after administration, commonly referred to as a ‘burst’. In some instances, this burst can result in an undesirable increase in the levels of biologically active agent leading to toxic effects and/or minimal release of agent thereafter providing sub-therapeutic concentration of the active agent. Therefore, a need still exists for providing sustained release parenteral depot compositions where a proper control over release kinetics by, for example, reducing the burst release of the active agent can be exerted and a continuous release of active agent for longer period of duration, for example, for a week or a month or for 3 months or more can be achieved, yet possessing good syringibility characteristics. Also, there is an unmet need for depot injectable compositions particularly for long-term use that are clinically tolerable, effective and safe, have a low potential for causing morbidity, and are cost-effective. Such compositions would highly improve patient compliance since they would abolish the need for daily administration of the drug for substantially long duration of treatment. The present invention provides novel in situ gelling depot or implant compositions which alleviates the limitations of the prior art.

### SUMMARY OF TIRE INVENTION

It is an objective of the present invention to provide novel injectable in-situ gelling depot or implant compositions exhibiting minimal burst release comprising at least one active agent(s) or its pharmaceutically acceptable salts, derivatives, isomers, polymorphs, solvates, hydrates, analogues, enantiomers, tautomeric forms or mixtures thereof, at least one biodegradable polymer(s), at least one viscosity enhancing agent(s) and optionally one or more pharmaceutically acceptable excipient(s), wherein the compositions are formulated as reconstitutable biodegradable microparticles or nanoparticles, and wherein the said compositions are in the form of a multi-component system preferably comprising at least two components, and wherein the said compositions provide a prolonged release of the active agent(s) for extended periods of time.

It is an objective of the present invention to provide novel injectable in situ gelling depot or implant compositions exhibiting minimal burst release of the active agent wherein said formulations exhibit a sustained release of an effective dose of the active agent for a period of at least one week and/or a reduction in release burst of the active agent compared to standard formulations when administered to a subject by parenteral route.

It is an objective of the present invention to provide novel injectable in situ gelling depot or implant compositions exhibiting minimal burst of the active agent which is achieved by the formation of a substantially cohesive gel-like mass due to gradual swelling of viscosity enhancing agent(s) in the aqueous physiological-type environment sufficient to form a solid or semisolid depot gel or implant shortly after the composition is administered into a living host.

It is an objective of the present invention to provide novel two component injectable in situ gelling depot or implant compositions exhibiting minimal burst release comprising at least one active agent(s) or its pharmaceutically acceptable salts, derivatives, isomers, polymorphs, solvates, hydrates, analogues, enantiomers, tautomeric forms or mixtures thereof from about 0.1% w/w to about 95% w/w, at least one biodegradable polymer(s) in an amount of from about 0.1% w/w to about 95% w/w, at least one viscosity enhancing agent(s) in an amount of from about 0.1% w/w to about 95% w/w and optionally one or more pharmaceutically acceptable excipient(s) in an amount of from about 0.1% to about 99.8% w/w based upon the total weight of the formulation, wherein the compositions are formulated as reconstitutable biodegradable microparticles or nanoparticles, and wherein the biodegradable polymer(s) is a polylactide polymer or a polyglycolide polymer or a poly(lactide-co-glycolide) co-polymer having an average molecular weight of from about 1,000 Daltons to about 200,000 Daltons, and wherein the said compositions provide a prolonged release of the active agent(s) for extended periods of time.

It is an objective of the present invention to provide novel injectable depot compositions comprising of at least two components, wherein component-1 is in the form of a readily dispersible composition preferably as microparticles or nanoparticles comprising at least one active agent(s) and at least one biodegradable polymer(s), optionally with one or more pharmaceutical acceptable excipient(s); and wherein component-2 is in the form of a liquid vehicle for reconstitution of component-1 comprising at least one water miscible or water immiscible solvent, optionally with one or more pharmaceutical acceptable excipient(s); and wherein the compositions comprise at least one viscosity enhancing agent(s) either present in component-1 or component-2 or both. The viscosity enhancing agent(s) is either present in component-1 or component-2 or both in an unhydrated form.

It is an objective of the present invention to provide novel injectable depot compositions comprising of at least two components, wherein component-1 is in the form of biodegradable microparticles or nanoparticles comprising at least one active agent(s), at least one biodegradable polymer(s), at least one viscosity enhancing agent(s) and optionally one or more pharmaceutical acceptable excipient(s); wherein the biodegradable microparticle or nanoparticle is partially or entirely embedded in the viscosity enhancing agent which acts as release modifier upon contact with body fluids by getting hydrated and forming a gel around the biodegradable microparticles.

It is an objective of the present invention to provide novel injectable depot compositions comprising of at least two components, wherein component-i is in the form of biodegradable microparticles or nanoparticles comprising at least one active agent(s), at least one biodegradable polymer(s), at least one viscosity enhancing agent(s) and optionally one or more pharmaceutical acceptable excipients; wherein the viscosity enhancing agent(s) is a biocompatible cellulosic polymer which acts as microparticle or nanoparticle stabilizer, active agent release modifier and/or a gel forming agent.

It is also an objective of the present invention to provide novel injectable depot compositions which provides a flowable composition for forming a solid or semi-solid biodegradable gel or implant in situ within a body, comprising at least one biodegradable polymer(s), at least one viscosity enhancing agent(s) and optionally at least one biocompatible solvent(s) that at least partially solubilizes the biodegradable polymer(s) and/or the viscosity enhancing agent(s) and is miscible or dispersible in aqueous body fluids, and capable of dissipating, diffusing or leaching from the composition into body fluid upon placement within a body, whereupon the biodegradable polymer(s) and/or the viscosity enhancing agent(s) coagulate or precipitate to form the gel or implant.

It is another objective of the present invention to provide process for preparation of such novel injectable compositions which comprises preparation of microparticles or nanoparticles comprising active agent(s) and a liquid vehicle in which the said microparticles or nanoparticles may be reconstituted prior to administration.

It is another objective of the present invention to provide a method of forming a depot gel or an implant in situ, in a living body, which comprises preparing an in situ gelling formulation according to the method described herein, placing the formulation within the body and allowing the liquid vehicle to disperse or dissipate to produce a solid or gel implant.

It is yet another objective of the present invention to provide a pharmaceutical kit suitable for in situ formation of a biodegradable depot gel or implant from the novel compositions as described herein, in the body of a subject in need thereof, which comprises a device containing microparticles of the active agent(s) and optionally one or more pharmaceutical acceptable excipient(s), and a device containing liquid vehicle and optionally one or more pharmaceutical acceptable excipient(s); wherein the devices allow for expulsion of the contents of the two devices for enabling mixing together prior to administration of contents into body of subject.

It is still another objective of the present invention to provide use of a depot in situ gelling or implant formulation as described herein in the manufacture of a medicament for the treatment of a condition treatable by the active agent in a mammal particularly a human being.

It is yet another objective of the present invention to provide a method of using the compositions according to the present invention which comprises administering to a subject/patient in need thereof an effective amount of the said composition.

Preferably the composition is administered to a subject particularly human or animal by injection, wherein the composition forms a drug depot that releases the pharmaceutically active agent(s) over a desired extended period of time, thereby increasing the efficacy of treatment associated with particularly chronic diseases, leading to greater patient compliance.

The novel compositions of the present invention preferably provide the active agent(s) to localize in certain tissues, thereby increasing the efficacy of treatment, associated with such tissues. The compositions of the present invention may be used for prophylaxis, amelioration or treatment of disease(s) or disorder(s) in a subject in need thereof.

### DETAILED DESCRIPTION OF THE INVENTION

The present invention provides novel injectable in situ gelling depot or implant compositions exhibiting minimal burst release comprising at least one active agent(s) or its pharmaceutically acceptable salts, derivatives, isomers, polymorphs, solvates, hydrates, analogues, enantiomers, tautomeric forms or mixtures thereof at least one biodegradable polymer(s), at least one viscosity enhancing agent(s) and optionally one or more pharmaceutically acceptable excipient(s), wherein the compositions are formulated as reconstitutable biodegradable microparticles or nanoparticles, and wherein the said compositions are in the form of a multi-component system preferably comprising at least two components, and wherein the said compositions provide a prolonged release of the active agent(s) for extended periods of time. The active agent wherever disclosed in the entire description hereinafter also encompasses its pharmaceutically acceptable salts, polymorphs, solvates, hydrates, analogues, enantiomers, tautomeric forms, derivatives or mixtures thereof unless otherwise mentioned. The term ‘reconstitutable’ used herein implies that the microparticles or nanoparticles are amenable to dispersion in an aqueous, hydro-alcoholic or oily liquid vehicle prior to administration.

In an embodiment, the present invention provides novel injectable in situ gelling depot or implant compositions exhibiting minimal burst wherein said formulation exhibits a sustained release of an effective dose of the active agent for a period of at least one week and/or a reduction in release burst of the active agent compared to standard formulations when administered to a subject by parenteral route.

In another embodiment, the present invention provides novel injectable in situ gelling depot or implant compositions exhibiting minimal burst release of the active agent which is achieved by the formation of a substantially cohesive gel-like mass due to gradual swelling of viscosity enhancing agent(s) in the aqueous physiological-type environment sufficient to form a solid or semisolid depot gel or implant shortly after the composition is administered into a living host. The compositions of the present invention comprises microparticles or nanoparticles of the active agent which gets embedded in the in situ gelled matrix formed upon in vivo administration; hence providing a dual mechanism for controlling the drug release i.e. the controlled release provided by the biodegradable polymer(s) and the gelled matrix formed due to the gelling of the viscosity enhancing polymer(s) upon contact with body fluids.

The present invention provides novel injectable compositions comprising at least one active agent(s), at least one biodegradable polymer(s), at least one viscosity enhancing agent(s) and optionally one or more pharmaceutically acceptable excipient(s), wherein the compositions are formulated as reconstitutable biodegradable microparticles or nanoparticles, and wherein the said compositions are in the form of a multi-component system preferably comprising at least two components namely component-1 and component-2.

In a preferred embodiment, the present invention provides novel two component injectable in situ gelling depot or implant compositions exhibiting minimal burst release comprising at least one active agent(s) or its pharmaceutically acceptable salts, derivatives, isomers, polymorphs, solvates, hydrates, analogues, enantiomers, tautomeric forms or mixtures thereof from about 0.1% w/w to about 95% w/w, at least one biodegradable polymer(s) in an amount of from about 0.1% w/w to about 95% w/w, at least one viscosity enhancing agent(s) in an amount of from about 0.1% w/w to about 95% w/w and optionally one or more pharmaceutically acceptable excipient(s) in an amount of from about 0.1% to about 99.8% w/w based upon the total weight of the formulation, wherein the compositions are formulated as reconstitutable biodegradable microparticles or nanoparticles, and wherein the biodegradable polymer(s) is a polylactide polymer or a polyglycolide polymer or a poly(lactide-co-glycolide) co-polymer having an average molecular weight of from about 1,000 Daltons to about 200,000 Daltons, and wherein the said compositions provide a prolonged release of the active agent(s) for extended periods of time.

In an embodiment of the present invention is provided novel injectable compositions comprising an active agent and at least one biodegradable polymer(s), wherein the ratio of active agent to the biodegradable polymer(s) is between about 1:100 to about 100:1.

Several drugs requiring medium to long term administration for prophylaxis and/or treatment of disease(s)/disorder(s) are presently available as oral dosage forms for daily administration. It is mandatory for the patients in need thereof to take the drugs daily to achieve desired therapeutic plasma concentrations for optimum therapeutic benefit. Patient compliance with such a daily dosing regimen is however, difficult to ensure, especially where the course of therapy is long or of intermediate or lifetime duration. Thus, there is a need for prolonged release formulations of such active agents to improve patient compliance/convenience and give patients optimum therapeutic benefit by abolishing the need to administer a dosage composition daily, which the present invention provides in the form of injectable compositions.

The novel injectable compositions of the present invention leads to less frequent dosing of drugs, and still provides an improved therapeutic effect with reduced side effects by effectively smoothening out the fluctuations in the plasma concentration-time profile. Most importantly, the prolonged release formulations of the present invention improves the ‘quality of life’ of patients undertaking long term treatment for chronic diseases/disorders such as cancers, psychosis, and the like.

The novel injectable composition of the present invention comprising effective dose of at least one active agent(s) is required to be administered in substantially low volumes which are convenient to administer and causes minimal pain on injection. Further the in situ gelling depot or implant compositions of the present invention are designed in such a manner so as to exhibit a gradual partitioning out of the depot during the depot formation stage upon in vivo administration thus leading to surprisingly low initial ‘burst’ release of the active agent. This in turn alleviates possibility of any side effects and enhances the ‘life’ of the depot in producing sustained release of the active agent for extended time duration.

The novel depot injectable compositions are able to provide a sustained release of the active agent for a prolonged duration even by using substantially low quantities of high molecular weight hydrophobic polymers such as the polylactide polymer or a polyglycolide polymer or a poly(lactide-co-glycolide) co-polymer thus resulting in less residual polymer remaining at the site of administration after the release of active core. Further, the compositions of the present invention are formulated such that the chances of dose dumping due to failure of the system is avoided or substantially reduced upon in vivo administration to a subject.

In a further embodiment, the novel depot injectable compositions of the present invention can comprise plurality of populations of microparticles or nanoparticles dispersed in a liquid vehicle constructed to release the active agent at different particular time intervals after formation of a depot comprising entrapped particles of active agent upon administration in vivo. According to an embodiment, the relative proportions of the biodegradable polymer(s) and the viscosity enhancing agent(s) can be varied to obtain different burst release time and amount of the active agent from the compositions of the present invention.

In an embodiment, the compositions of the present invention upon in vivo administration forms a substantially homogeneous, sponge-like gel or an implant, which retain their gel-like consistency over a longer period than do prior art devices and permit the delivery of the active agent over a prolonged period. Furthermore, the surface pores of the depot gel or implant offer only a limited opportunity for water from body fluids to enter the implant immediately after implantation, thus controlling the burst effect.

If the polymer composition is to be administered as an injectable gel, the level of polymer dissolution will need to be balanced with the resulting gel viscosity, to permit a reasonable force to dispense the viscous gel from a needle, and the potential burst effect. Highly viscous gels enable the beneficial agent to be delivered without exhibiting a significant burst effect, but may make it difficult to dispense the gel through a needle. Therefore the compositions of the present invention are designed comprising a viscosity enhancing agent(s) is present in an unhydrated form such that it does not swell or make the injectable composition undesirably viscous during reconstitution prior to administration thus permitting easy syringibility through a needle. At the same time, the undesirable initial ‘burst’ release of the active agent is prevented or substantially minimized since the unhydrated viscosity enhancing agent(s) in the injectable composition after in vivo administration gets hydrated gradually and swells to form a gel-like cohesive mass through which the active agent is gradually released thus leading to the prolongation of the duration of drug release.

In an embodiment, the compositions according to the present invention do not have an isolated initial release burst but rather a gradual release at the start which stabilizes regularly towards the necessary and sufficient sustained release profile (circulating level). The continuity in the release of the active agent in vivo represents an important advantage of this type of formulation as the dose circulating in the patient can thus be maintained at sufficient levels in order to obtain a therapeutic effect and the circulating active agent concentration will remain greater than or equal to the requirements of the treatment without an initial burst, and without peaks or troughs. The inventors of the present invention have thus discovered that the use of formulations having these release profile characteristics made it possible to increase the treatment intervals and to alleviate frequent dosing requirements thus improving patient compliance especially for treatment of medium to long term pathological conditions.

In an embodiment, the present invention provides novel injectable compositions comprising an active agent, at least one biodegradable polymer(s) and optionally one or more pharmaceutically acceptable excipient(s), wherein the compositions are formulated as biodegradable microparticles or nanoparticles which can be reconstituted with an aqueous, hydro-alcoholic or oily liquid vehicle prior to administration. The novel compositions are in the form of an in situ gelling composition or an implant composition which form a depot upon administration in vivo upon contact with body fluids therefore providing a prolonged release of the active agent for extended periods of time. The novel compositions of the present invention are capable of producing a prolonged release of the active agent for at least 7 days preferably for a period of at least 15 days to 6 months, or more.

In accordance with as aspect of the present invention is provided novel injectable depot compositions comprising of at least two components, wherein component-1 is in the form of a readily dispersible composition preferably as microparticles or nanoparticles comprising at least one active agent(s) and at least one biodegradable polymer(s), optionally with one or more pharmaceutical acceptable excipient(s); and wherein component-2 is in the form of a liquid vehicle for reconstitution of component-1 comprising at least one water miscible or water immiscible solvent optionally with one or more pharmaceutical acceptable excipient(s); and wherein the compositions comprise at least one viscosity enhancing agent(s) either present in component-1 or component-2 or both. The viscosity enhancing agent(s) is either present in component-1 or component-2 or both in an unhydrated form.

In another embodiment the present invention provides injectable depot compositions comprising of at least two components, wherein component-1 is in the form of biodegradable microparticles or nanoparticles comprising at least one active agent(s), at least one biodegradable polymer(s), at least one viscosity enhancing agent(s) and optionally one or more pharmaceutical acceptable excipient(s); wherein the biodegradable microparticles or nanoparticles are partially or entirely embedded in the viscosity enhancing agent which acts as release modifier upon contact with body fluids by getting hydrated and forming a gel around the biodegradable microparticles. In an aspect, the viscosity enhancing agent(s) is a biocompatible cellulosic polymer which acts as microparticle or nanoparticle stabilizer, active agent release modifier and/or a gel forming agent.

In an embodiment the novel injectable depot compositions comprise of at least two component system, wherein component-1 comprises a readily dispersible composition preferably in the form of microparticles or nanoparticles which comprise at least one active agent(s) and at least one biodegradable polymer(s) optionally with channel forming agent(s) to form biodegradable microparticles or nanoparticles having desired drug release characteristics; and wherein component-2 is a liquid vehicle for reconstituting the component-1; and wherein the compositions comprise at least one viscosity enhancing agent(s) either present in component-1 or component-2 or both; and wherein the composition forms an in situ gel preferably at the site of injection upon contact with body fluids.

The present invention provides novel injectable depot compositions which are flowable and which are capable of forming a solid or semi-solid biodegradable gel or implant in situ within a body. In another embodiment, the present invention provides an in situ gelling composition comprising the active agent and a PLGA polymer, dissolved dispersed or suspended in suitable liquid vehicle such as an aqueous vehicle or an oily vehicle. The compositions of the invention, upon contact with water or bodily fluids, result in the precipitation of both the polymer and the active agent and subsequent formation of a gel or an implant within which the active agent is incorporated. The active agents subsequently diffuse from the gel or implant over an extended period of time to provide the desired pharmacological effect. In still other embodiments, the active agent may be encapsulated or otherwise incorporated into particles, such as microspheres, nanospheres, liposomes, lipospheres, micelles, and the like, or it may be conjugated to a polymeric carrier. In another embodiment, the microparticles or nanoparticles of the active agent useful for formulating the injectable composition are produced by a method which comprises spray-drying a solution or suspension comprising the active agent. In yet another embodiment, the injectable composition of the present invention comprising microparticles or nanoparticles can be delivered through a parenteral, transdermal, transmucosal or subcutaneous route using a needleless syringe.

The active agent of the present invention is selected from but not limited to a group comprising adrenergic agent; adrenocortical steroid; adrenocortical suppressant; alcohol deterrent; aldosterone antagonist; amino acid; anabolic; analeptic; analgesic; androgen; anesthetic; anorectic; anterior pituitary suppressant; antheimintic; antiacne agent; anti-adrenergic; anti-allergic; anti-amebic; anti-androgen; anti-anemic; antianginal; anti-anxiety; anti-arthritic; anti-asthmatic; anti-atherosclerotic; antibacterial; anticholelithic; anticholelithogenic; anticholinergic; anticoagulant; anticoccidal; anticonvulsant; antidepressant; antidiabetic; antidiarrheal; antidiuretic; antidote; anti-emetic; anti-epileptic; anti-estrogen; antifibrinolytic; antifungal; antiglaucoma agent; antihemophilic; antihemorrhagic; antihistamine; antihyperlipidemia; antihyperlipoproteinemic; antihypertensive; antihypotensive; anti-infective, anti-inflammatory; antimicrobial; antimigraine; antimycotic, antinauseant, antineoplastic, antineutropenic, antiobessional agent; antiparasitic; antiparkinsonian; antiperistaltic, antipneumocystic; antiproliferative; antiprostatic hypertrophy; antiprotozoal; antipruritic; antipsychotic; antirheumatic; antischistosomal; antisecretory; antispasmodic; antithrombotic; antitussive; anti-ulcerative; anti-urolithic; antiviral; benign prostatic hyperplasia therapy agent; blood glucose regulator; bone resorption inhibitor; bronchodilator; carbonic anhydrase inhibitor; cardiac depressant; cardioprotectant; cardiotonic; cardiovascular agent; choleretic; cholinergic agonist; cholinesterase deactivator; coccidiostat; cognition enhancer; depressant; diuretic; dopaminergic agent; enzyme inhibitor; estrogen; fibrinolytic; fluorescent agent; free oxygen radical scavenger; gastrointestinal motility effector; glucocorticoid; gonad-stimulating principle; hair growth stimulant; hemostatic; histamine H2 receptor antagonists; hormone; hypocholesterolemic; hypoglycemic; hypolipidemic; hypotensive; immunizing agent; immunomodulator; immunoregulator; immunostimulant; immunosuppressant; impotence therapy adjunct; inhibitor; keratolytic; LNRH agonist; luteolysin; memory adjuvant; mental performance enhancer; mood regulator; mucolytic; mydriatic; nasal decongestant; neuromuscular blocking agent; neuroprotective; NMDA antagonist; non-hormonal sterol derivative; oxytocic; plasminogen activator; platelet activating factor antagonist; platelet aggregation inhibitor; post-stroke and post-head trauma treatment; potentiator; progestin; prostaglandin; prostate growth inhibitor; prothyrotropin; psychotropic; pulmonary surface; radioactive agent; regulator; relaxant; repartitioning agent; sclerosing agent; sedative; sedative-hypnotic; selective adenosine A1 antagonist; serotonin antagonist; serotonin inhibitor; serotonin receptor antagonist; steroid; symptomatic multiple sclerosis; thyroid hormone; thyroid inhibitor; thyromimetic; tranquilizer; treatment of amyotrophic lateral sclerosis; treatment of cerebral ischemia; treatment of Paget's disease; treatment of unstable angina; uricosuric; vasoconstrictor; vasodilator; xanthine oxidase inhibitor and their pharmaceutically acceptable salts, esters, amides, polymorphs, solvates, hydrates, analogues, enantiomers, tautomeric forms, metabolites or mixtures thereof, used either alone or in combination thereof.

Preferably the active agent of the present invention is an antineoplastic agent selected from but not limited to a group comprising antineoplastic drugs, monoclonal antibodies, immunotherapy or radiotherapy or biological response modifiers. Suitable biological response modifiers include lymphokines and cytokines such as interleukins, interferons alpha, beta, or delta and Tumor Necrosis Factor (TNF). Other chemotherapeutic agents which are useful in the treatment of disorders due to abnormal cell proliferation include alkylating agents, for instance nitrogen mustards such as mechlorethamine, cyclophosphamide, melphalan and chlorambucil; tamsulosin; alkyl sulphonates such as busulfan; nitrosoureas such as carmustine, lomustine, semustine and streptozocin; triazenes such as dacarbazine; antimetabolites such as folic acid analogues, for instance methotrexate; pyrimidine analogues such as fluorouracil and cytarabine; purine analogues such as mercaptopurine and thioguanine; taxanes such as paclitaxel, docetaxel or PNU-1; natural products, for instance vinca alkaloids such as vinblastine, vincristine and vindesine; epipodophyllotoxins such as etoposide and teniposide; antibiotics such as dactinomycin, daunorubicin, doxorubicin, bleomycin, plicamycin and mitomycin; enzymes such as L-asparaginase; various agents such as coordination complexes of platinum, for instance cisplatin; substituted ureas such as hydroxyurea; methyl-hydrazine derivatives such as procarbazine; adrenocortical suppressants such as mitotane and aminoglutethimide; hormones and antagonists such as adrenocortico-steroids e.g. prednisone; progestins such as hydroxyprogesterone caproate, methoxyprogesterone acetate and megestrol acetate; oestrogens such as diethylstilboestrol and ethynyloestradiol; antioestrogens such as tamoxifen and anastrozole; and androgens such as testosterone propionate and fluoxymesterone; antidepressants such as ziprasidone; antipsychotics such as risperidone, or their pharmaceutically acceptable salts, hydrates, polymorphs, esters, and derivatives, used either alone or in combination thereof.

In an embodiment of the present invention, novel injectable depot compositions comprise of at least two component system, wherein component-1 comprises a readily dispersible composition preferably in the form of microparticles which comprise at least one antineoplastic agent(s), preferably aromatase inhibitors, more preferably anastrozole or its salts, polymorphs, solvates, hydrates, analogues, enantiomers, tautomeric forms, derivatives or mixtures thereof, as active agent either alone or in combination with other active agent(s) and at least one biodegradable polymer, optionally with at least one channel forming agent(s); and wherein component-2 is an aqueous, hydro-alcoholic or oily liquid vehicle for reconstituting the component-1; and wherein the compositions comprise at least one viscosity enhancing agent(s) either present in component-1 or component-2 or both.

In an embodiment of the present invention, novel injectable depot compositions comprise of at least two component system, wherein component-1 comprises a readily dispersible composition preferably in the form of microparticles which comprise at least one antipsychotic(s) such as risperidone or donepezil or its salts, polymorphs, solvates, hydrates, analogues, enantiomers, tautomeric forms, derivatives or mixtures thereof, as active agent either alone or in combination with other active agent(s) and at least one biodegradable polymer(s), optionally with at least one channel forming agent(s); and wherein component-2 is an aqueous, hydro-alcoholic or oily liquid vehicle for reconstituting the component-1; and wherein the compositions comprise at least one viscosity enhancing agent(s) either present in component-1 or component-2 or both.

In an embodiment of the present invention, the biodegradable polymer is selected from but not limited to a group comprising lactic acid-based polymers such as polylactides e.g. poly(D,L-lactide) i.e. PLA; glycolic acid-based polymers such as polyglycolides (PGA) e.g. Lactel® from Durect; poly(D,L-lactide-co-glycolide) i.e. PLGA, (Resomer® RG-504, Resomer® RG-502, Resomer® RG-504H, Resomer® RG-502H, Resomer® RG-504S, Resomer® RG-502S, from Boehringer, Lactel® from Durect); polycaprolactones such as Poly(e-caprolactone) i.e. PCL (Lactel® from Durect); polyanhydrides; poly(Sebacic acid) SA; poly(Ricenolic acid) RA; poly(Fumaric acid), FA; poly(Fatty acid dimmer), FAD; poly(terephthalic acid), TA; poly(isophthalic acid), IPA; poly(p-{carboxyphenoxy}methane), CPM; poly(p-{carboxyphenoxy}propane), CPP; poly(p-{carboxyphenoxy}hexane), CPH; polyamines, polyurethanes, polyesteramides, polyorthoesters {CHDM: Cis/trans-cyclohexyl dimethanol, HD:1,6-hexanediol. DETOU: (3,9-diethylidene-2,4,8,10-tetraoxaspiro undecane)}; polydioxanones; polyhydroxybutyrates; polyalkyene oxalates; polyamides; polyesteramides; polyurethanes; polyacetals; polyketals; polycarbonates; polyorthocarbonates; polysiloxanes; polyphosphazenes; succinates; hyaluronic acid; poly(malic acid); poly(amino acids); polyhydroxyvalerates; polyalkylene succinates; polyvinylpyrrolidone; polystyrene; synthetic celluloses; polyacrylic acids; polybutyric acid; polyvaleric acid; polyethylene glycol; polyhydroxycellulose; chitin; chitosan; polyorthoesters and copolymers, terpolymers; dimethyl isosorbide; lipids such as cholesterol, lecithin; poly(glutamic acid-co-ethyl glutamate) and the like, or mixtures thereof.

Preferably the biodegradable polymer is a lactic acid-based polymer, more preferably polylactide, or poly(D,L-lactide-co-glycolide) i.e. PLGA. Preferably the biodegradable polymer is present in an amount between about 10% to about 98% w/w of the component-1. The lactic acid-based polymer has a monomer ratio of lactic acid to glycolic acid in the range of 100:0 to about 0:100 preferably 100:0 to about 10:90 and has an average molecular weight of from about 1,000 to 200,000 daltons. It might be emphasized that the choice and the quantity of biodegradable polymer is governed by the nature and quantity of active agent used, the desired particle size of the composition, the intended use, the duration of use, and the like. In another embodiment, the component-1 of the present invention additionally comprises excipients selected from but not limited to a group comprising channel forming agents, oily components, emulsifiers, preservatives, antioxidants, stabilizers or mixtures thereof.

In another embodiment of the present invention, a process of preparation of microparticles or nanoparticles involves preferably o/w emulsion technique followed by solvent evaporation. The microparticles or nanoparticles comprise an oil phase wherein the oil phase is selected from but not limited to a class of water immiscible solvents preferably having low boiling point such as esters (e.g. ethyl acetate, butyl acetate), halogenated hydrocarbons (e.g. dichloromethane, chloroform, carbon tetrachloride, chloroethane, dichloroethane, trichloroethane), ethers (e.g. ethyl ether, isopropyl ether), aromatic hydrocarbons (e.g. benzene, toluene, xylene), carbonates (e.g. diethyl carbonate), or the like or mixtures thereof Suitable emulsifiers are used in the preparation of the microparticles or nanoparticles to enhance the stabilization of oil droplets against coalescence, wherein the emulsifier is selected from but not limited to a group comprising polyoxyethylene sorbitan fatty acid esters e.g. mono- and tri-lauryl, palmityl, stearyl and oleyl esters; sorbitan fatty acid esters (SPAN®); polysorbates (Tween®), polyvinyl alcohol, polyvinyl pyrrolidone, gelatin, lecithin, polyoxyethylene castor oil derivatives (Cremophor®), particularly suitable are polyoxyl 35 castor oil (Cremophor®EL) and polyoxyl 40 hydrogenated castor oil (Cremophor®RH40); tocopherol; tocopheryl polyethylene glycol succinate (vitamin E TPGS); tocopherol palmitate and tocopherol acetate; Polyoxyethylene-polyoxypropylene co-polymers (Pluronic® or Poloxamer®), and the like or mixtures thereof. Suitable channel forming agents optionally used to formulate the microparticles or nanoparticles is selected from but not limited to a group comprising polyglycols, ethyl vinyl alcohols, glycerin, pentaerythritol, polyvinyl alcohols, polyvinyl pyrrolidone, vinyl pyrrolidone, N-methyl pyrrolidone, polysaccharides such as dextrines and/or hydrolyzed starch, saccharides, sugar alcohols and the like, or mixtures thereof.

In an embodiment of the present invention, the viscosity enhancing agent of component-1 is selected from but not limited to group comprising cellulose derivatives, such as hydroxypropyl cellulose, hydroxyethyl cellulose, hydroxypropyl methyl cellulose, methylcellulose, sodium carboxymethyl cellulose and its derivatives, vinyl polymers, polyoxyethylene-polyoxypropylene polymers or co-polymers (Pluronics®), polysaccharides such as glycosaminoglycans, agar, pectin, alginic acid, dextran, starch and chitosan, proteins, poly(ethyleneoxide), acrylamide polymers, polyhydroxy acids, polyanhydrides, polyorthoesters, polyamides, polycarbonates, polyalkylenes, polyalkylene glycols, polyalkylene oxides, polyalkylene terepthalates, polyvinyl alcohols such as polyacrylic acid, polymethacrylic acid, polyvinyl pyrrolidone and polyvinyl alcohol, polyvinyl ethers, polyvinyl esters, polyvinyl halides, polyvinylpyrrolidone, polysiloxanes, polyvinyl acetates, polystyrene, polyurethanes, synthetic celluloses, polyacrylic acids, polybutyric acid, polyvaleric acid, poly(lactide-co-caprolactone), and copolymers, derivatives, and the like; or mixtures thereof Preferably the viscosity enhancing agent(s) is a high viscosity grade of sodium carboxymethyl cellulose or methyl cellulose. Preferably viscosity enhancing agent is present in an amount between about 0.1% to about 50%, more preferably between about 0.5% to about 50% by weight of the composition either in component-1 or component-2 or both.

In another embodiment of the present invention, the liquid vehicle (of component-2) is in the form of an aqueous vehicle comprising water and optionally water miscible solvent selected from but not limited to group comprising preferably a water-miscible alcohol, for example, methanol, ethanol, n-propyl alcohol, isopropyl alcohol, tert-butyl alcohol, ethylene glycol or propylene glycol; dimethylsulfoxide; dimethylformamide; a water-miscible ether, for example tetrahydrofuran; a water-miscible nitrile, for example acetonitrile; a water-miscible ketone, for example acetone or methyl ethyl ketone; an amide, for example dimethylacetamide; propylene glycol; glycerin; polyethylene glycol 400; glycofurol (tetraglycol), and the like; or mixtures thereof. Preferably the water miscible solvent useful in the present invention is selected from glycerin, ethanol, propylene glycol, polyethylene glycols, or mixtures thereof.

In another embodiment of the present invention, the liquid vehicle of the present invention is an oily vehicle comprising at least one oily component selected from but not limited to a group comprising vegetable oils such as corn oil, almond oil, sunflower oil, peanut oil, olive oil, castor oil, soybean oil, safflower oil, cottonseed oil, and the like, or a lipophilic compound such as an ester of a medium chain fatty acid, an ester of a long chain fatty acid, dimethyl isosorbide, and the like; optionally with a surfactant selected from a group comprising anionic, cationic, non-ionic or zwitterionic surfactants and/or one or more other pharmaceutically acceptable excipient(s). It might be emphasized that when the liquid vehicle (of component-2) is in the form of aqueous vehicle, then the viscosity enhancing agent is preferably present in component-2 and when the liquid vehicle (of component-2) is in the form of oily vehicle, then the viscosity enhancing agent is preferably present in component-1. In another embodiment of the present invention, the water immiscible solvent of component-2 is selected from but not limited to group comprising ethyl acetate, diethyl ether, hexane, toluene, isopropyl acetate, dichloromethane, chloroform, and the like; or mixtures thereof. In another embodiment of the present invention, the liquid vehicle of the present invention is comprising of at least one fluorocarbons component selected from but not limited to a group comprising perfluorocarbons such as perfluorooctane, perfluorohexane, perfluorodecane and the like; volatile anaesthetics such as sevoflurane, desflurane, isoflurane, and the like; or mixtures thereof.

In an embodiment, the component-2 of the present invention additionally comprises of one or more substances selected from but not limited to a group comprising co-surfactants, solvents/co-solvents, water, oily component, hydrophilic solvents, preservatives, antioxidants, anti-foaming agents, stabilizers, buffering agents, pH adjusting agents, osmotic agents, isotonicity producing agents, or any other excipient soluble in the water miscible solvent known to the art or mixtures thereof In an embodiment of the present invention, the co-surfactant is selected from but not limited to a group comprising polyethylene glycols; polyoxyethylene-polyoxypropylene block copolymers known as “poloxamer”; polyglycerin fatty acid esters such as decaglyceryl monolaurate and decaglyceryl monomyristate; sorbitan fatty acid ester such as sorbitan monostearate; polyoxyethylene sorbitan fatty acid ester such as polyoxyethylene sorbitan monooleate(TWEEN®); polyethylene glycol fatty acid ester such as polyoxyethylene monostearate; polyoxyethylene alkyl ether such as polyoxyethylene lauryl ether; polyoxyethylene castor oil and hardened castor oil, such as polyoxyethylene hardened castor oil; and the like or mixtures thereof In an embodiment of the present invention, the solvent/cosolvent is selected from but not limited to a group comprising alcohols such as propylene glycol, polypropylene glycol, polyethylene glycol (such as PEG300, 400, 600, etc.), glycerol, ethanol, triacetin, dimethyl isosorbide, glycofurol, propylene carbonate, water, dimethyl acetamide, and the like or mixtures thereof More preferably the solvent used is ethanol. The choice of the solvent/cosolvent and its quantity primarily depends on the solubility of the active agent(s). It might be emphasized that when the composition is formulated with a water-soluble solvent such as ethanol, the solvent will diffuse rapidly out of the injected volume leaving a high viscosity depot that is well suited for long term drug delivery. Suitable anti-foaming agents include for example silicon emulsions or sorbitan sesquoleate. Suitable stabilizers to prevent or reduce the deterioration of the other components in compositions of the present invention include antioxidants such as glycine, alpha-tocopherol or ascorbate, BHA, BHT, and the like or mixtures thereof. Suitable tonicity modifier includes for example mannitol, sodium chloride, and glucose. Suitable buffering agent includes fore example acetates, phosphates, and citrates with suitable cations. It might be however understood that certain excipients used in the present composition can serve more than one purpose.

In an embodiment, the present invention provides a pharmaceutical kit suitable for in situ formation of a biodegradable depot gel or implant from the novel compositions as described herein, in the body of a subject in need thereof, which comprises a device containing the active agent microparticles and optionally one or more pharmaceutical acceptable excipient(s), and a device containing liquid vehicle and optionally one or more pharmaceutically acceptable excipient(s); wherein the devices allow for expulsion of the contents of the two devices for enabling mixing together prior to administration of the contents into the body of the subject.

In an embodiment, the present invention provides novel injectable depot compositions wherein the component-1 is presented as a dry powder and component-2 is presented as a liquid vehicle. The said component-1 is reconstituted with component-2 to obtain a parenteral suspension, which when injected intramuscularly or subcutaneously, forms a hydrogel at injection site that acts as a depot from which the active agent(s) is released in a sustained manner for prolonged time period. This helps in simplifying the available daily dosage regimen for the active agent(s). Further, the primary barrier for the release of the active agent(s) would be the in situ hydrogel formed and the secondary barrier for release of the active agent(s) would be anticipated from the biodegradable polymeric drug microparticles or nanoparticles that leads to an effective depot for the active agent(s) at the injection site and releases the active agent(s) in a sustained manner over an extended period of time to achieve the desired therapeutic concentration. It is an advantage of the present invention that rate of release of the active agent(s) can be dually modulated by in situ gelling composition and the biodegradable particulate form of the active agent(s) dispersed in the gelling composition. The term “in situ gelling composition” as used herein refers to a composition comprising a drug preferably as microparticles or nanoparticles, a biodegradable polymer and optionally a viscosity enhancing agent, which is optionally reconstituted with a liquid vehicle and delivered to a patient as an injectable liquid but solidifies into a solid depot composition upon in vivo administration.

In another embodiment, the component-2 of the present invention comprises of one or more water miscible solvents or cosolvents which can get easily assimilated away from the injection site by the bodily process leaving behind the polymeric gel material at the injection site. In another aspect of the present invention, the composition of component-2 shall preferably keep the viscosity building polymeric material in anhydrate particulate form; thus preventing a viscosity build up in reconstituted suspension for injection, which in turn facilitates syringibility even at higher concentration of high viscosity building polymers used in the formulation.

In an embodiment, component-1 of the two component system relates to biodegradable microparticles or nanoparticles formulated as matrix system comprising an active agent(s), at least one biodegradable polymer(s), at least one hydrophilic cellulosic biocompatible polymer(s) entrapped between the biodegradable microparticles or nanoparticles matrix system acting as release modifier; and optionally one or more pharmaceutical excipient(s), wherein the hydrophilic cellulosic biocompatible polymer upon contact with bodily fluids gets hydrated faster and forms a gel around the biodegradable microparticles or nanoparticles and later on further hydration leads the gel layer to erode followed by dissolution of hydrated entrapped cellulosic biocompatible polymer leading to formation of channels in the biodegradable microparticles or nanoparticles matrix through which drug is released. Also there is a biodegradation of microparticles or nanoparticles. This leads to an advantage of reducing the time of production of microparticles or nanoparticles by removing manufacturing steps like washing and filtration/centrifugation steps. The present invention also describes a novel method of preparation of biodegradable microparticles or nanoparticles without using parenterally unacceptable emulsion stabilizer such as polyvinyl alcohol (PVA). Component-1 forms a readily dispersible composition upon reconstitution with suitable liquid vehicle i.e., component-2. In an embodiment the component-2 is in the form of preferably liquid vehicle for reconstitution of component-1 comprising at least one water immiscible solvent (e.g., oil) and optionally with one or more pharmaceutical acceptable excipients. In another preferred embodiment the component-2 is in the form of preferably liquid vehicle for reconstitution of component-1 comprising at least one oil, at least one surfactant and optionally with one or more pharmaceutical acceptable excipient(s). In one of the embodiment the component-2 is in the form of a liquid vehicle for reconstitution of component-1 comprising at least one water miscible solvent, optionally with one or more excipient(s).

The present invention also describes a novel method of preparation of biodegradable microparticles or nanoparticles in the form of matrix by using a cellulosic biocompatible polymer having multiple properties like emulsion stabilizer, drug release modifier and a gel forming agent. In an embodiment, a cellulosic polymer such as sodium carboxymethyl cellulose (NaCMC) is used as an emulsion stabilizer during preparation of the microparticles or nanoparticles and entraps the individual microparticles or nanoparticles formed. The said polymer is approved for parenteral use and hence does not need removal. The said polymer also acts as a viscosity enhancing agent.

In an embodiment of the present invention, temperature sensitive biocompatible polymers may be used as the gel matrix, for example, a block copolymer having thermal gelation properties wherein the polymer is a gel at physiological temperatures (about 37° C.) and is a liquid above or below physiological temperatures would be functional. In the case of a gel having reverse thermal-gelation properties, the block copolymer would be a liquid at temperatures below the gelation temperature and would form a gel at above the gelation temperature. Conversely, a block copolymer having conventional thermal-gelation properties would be a liquid above the gelation temperature and a gel at or below the gelation temperature. When a biocompatible block copolymer having reverse thermal-gelation properties is employed, microparticles containing tamsulosin or letrozole can be loaded in the block copolymer at below physiological temperatures such as room temperature. Because such block copolymers are soluble in water when cooled, the microparticles or nanoparticles may be easily loaded within the solution. Furthermore, when administered, the block copolymer solution, once in the gel state, is able to retain the microparticles or nanoparticles.

In another embodiment, the viscosity enhancing agent(s) present in the composition of present invention is partially or entirely entrapped in the biodegradable microparticles or nanoparticles and acts as release modifier upon contact with bodily fluids by getting hydrated and forming a gel around the biodegradable microparticles or nanoparticles. In an embodiment, the viscosity enhancing agent(s) is a biocompatible cellulosic polymer which acts as microparticle or nanoparticle stabilizer, active agent release modifier and/or a gel forming agent. The compositions of the present provides lower rate of release shortly after the formation of depot or implant after the injection i.e. a low “initial burst” since a higher “initial burst” can result in an undesirable increase in the levels of biologically active agent leading to toxic effects and/or minimal release of agent thereafter providing sub-therapeutic concentration of the active agent thereby making the composition unsuitable for prolonged duration. To illustrate the novel injectable depot compositions of the present invention, the inventors of the present invention have now developed an improved composition comprising anastrozole as the active agent

A pharmacokinetic study was conducted in female rabbits using four anastrozole i.m. depot compositions (compositions disclosed hereinafter in examples 1 to 4 and referred to as F-1, F-2, F-3 and F4 respectively). A single dose of 5 mg/kg was administered i.m. and concentration of anastrozole (ng/ml) in plasma was estimated by using LC/MS for 60 days (for F-3 and F4) and for 10 days (for F-1 and F-2). 16 rabbits in 4 groups were used for studying the formulations F-1, F-2, F-3 and F4. The blood was withdrawn at the following time interval: 0, 1, 2, 4, 8 and 12 hr on day 1, 0 and 8 hr on day 2 to day 7, and 0 hr on day 8, 9, 10, 13, 14, 16, 18, 21, 24, 27, 30, 35, 40, 45, 50 & 60. The pharmacokinetic (PK) parameters particularly the Cmax for all dose groups were estimated and AUC (initial i.e. AUC_{0-1day} and at 60 days i.e. AUC_{0-60days}) was calculated for F-3 and F-4. The data is presented as Table-1, Table-2 and Table-3.

TABLE 1
Mean Concentration (ng/ml) versus Time (hr) Data for F-3 and F-4

| | Actual | Cumulative | F-3 | F-4 |
|---|---|---|---|---|
| Day | Time (hr) | time (hr) | Mean ± SD | Mean ± SD |
| Day 1 | 0 | 0 | 0 ± 0 | 0 ± 0 |
| | 1 | 1 | 75.02 ± 1.26 | 73.6 ± 1.69 |
| | 2 | 2 | 80.02 ± 2.45 | 77.2 ± 1.52 |
| | 4 | 4 | 110.42 ± 16.88 | 78.2 ± 3.29 |
| | 8 | 8 | 119.35 ± 18.52 | 79.75 ± 8.95 |
| | 12 | 12 | 113.75 ± 21.28 | 80.67 ± 4.03 |
| Day 2 | 0 | 24 | 84.1 ± 29.32 | 75.77 ± 10.32 |
| | 8 | 32 | 55.27 ± 5.98 | 53.7 ± 3.79 |
| Day 3 | 0 | 48 | 36.95 ± 19.66 | 33.3 ± 5.43 |
| | 8 | 56 | 25.53 ± 23.51 | 27 ± 9.42 |
| Day 4 | 0 | 72 | 19.8 ± 14.86 | 22.12 ± 7.82 |
| | 8 | 80 | 16.06 ± 11.57 | 16.62 ± 4.71 |
| Day 5 | 0 | 96 | 15.59 ± 9.50 | 14.6 ± 6.07 |
| | 8 | 104 | 12.03 ± 4.12 | 14.1 ± 0.67 |
| Day 6 | 0 | 120 | 13.32 ± 1.47 | 13.02 ± 1.54 |
| | 8 | 128 | 12.75 ± 0.36 | 12.42 ± 0.68 |
| Day 7 | 0 | 144 | 11.4 ± 1.32 | 11.62 ± 0.49 |
| | 8 | 152 | 10.97 ± 0.28 | 10.65 ± 0.42 |
| Day 8 | 0 | 168 | 12.35 ± 0.52 | 11.45 ± 1.10 |
| Day 9 | 0 | 192 | 12.5 ± 1.06 | 15.47 ± 2.10 |
| Day 10 | 0 | 216 | 7.60 ± 8.44 | 6.27 ± 2.86 |
| Day 13 | 0 | 288 | 2.50 ± 2.09 | 5.25 ± 2.36 |
| Day 14 | 0 | 312 | 4.76 ± 4.20 | 5.62 ± 0.84 |
| Day 16 | 0 | 360 | 7.29 ± 4.89 | 5.31 ± 1.17 |
| Day 18 | 0 | 408 | 5.42 ± 4.39 | 4.66 ± 1.41 |
| Day 21 | 0 | 480 | 5.59 ± 3.79 | 4.11 ± 0.77 |
| Day 24 | 0 | 552 | 3.94 ± 3.24 | 2.52 ± 0.34 |
| Day 27 | 0 | 624 | 3.88 ± 2.71 | 2.62 ± 0.17 |
| Day 30 | 0 | 696 | 3.43 ± 2.54 | 1.86 ± 0.20 |
| Day 35 | 0 | 816 | 3.04 ± 2.56 | 2.45 ± 0.41 |
| Day 40 | 0 | 936 | 2.93 ± 2.29 | 2.52 ± 0.61 |
| Day 45 | 0 | 1056 | 2.01 ± 1.38 | 2.43 ± 0.76 |
| Day 50 | 0 | 1176 | 1.91 ± 1.30 | 1.99 ± 0.32 |
| Day 60 | 0 | 1416 | 4.24 ± 3.55 | 3.67 ± 1.30 |

TABLE 2
PK Data for F-3 and F-4 (Mean ± SD)

| PK parameter | F-3 | F-4 |
|---|---|---|
| Cmax (ng/ml) | 116.8 ± 21.23 | 84.03 ± 4.39 |
| AUC_{0-60 days} (hr*ng/ml) | 10583.4 ± 5029.0 | 9323.2 ± 1046.1 |
| AUC_{0-1 day} (hr*ng/ml) | 2276.4 ± 425.2 | 1843.1 ± 117.9 |
| % burst [(AUC_{0-1 day}/ | 21.51 | 19.77 |
| AUC_{0-60 days}) × 100] | | |

TABLE 3
Mean Concentration (ng/ml) versus Time (hr) Data

| | Actual | Cumulative | F-1 | F-2 |
|---|---|---|---|---|
| Day | Time (hr) | time (hr) | Mean ± SD | Mean ± SD |
| Day 1 | 0 | 0 | 0 ± 0 | 0 ± 0 |
| | 1 | 1 | 72.75 ± 19.46 | 55.1 ± 23.05 |
| | 2 | 2 | 91.72 ± 26.86 | 82.92 ± 30.36 |
| | 4 | 4 | 97.27 ± 29.02 | 106.72 ± 36.77 |
| | 8 | 8 | 172.75 ± 40.92 | 123 ± 21.96 |
| | 12 | 12 | 154.5 ± 37.81 | 106.07 ± 24.66 |
| Day 2 | 0 | 24 | 124.27 ± 28.49 | 75.1 ± 12.96 |
| | 8 | 32 | 56.15 ± 3.82 | 47.1 ± 8.76 |
| Day 3 | 0 | 48 | 37.55 ± 16.26 | 27.05 ± 5.86 |
| | 8 | 56 | 37.67 ± 28.52 | 16.77 ± 2.36 |
| Day 4 | 0 | 72 | 30.72 ± 30.63 | 14.62 ± 0.53 |
| | 8 | 80 | 21.6 ± 17.19 | 12.8 ± 0.24 |
| Day 5 | 0 | 96 | 18.98 ± 16.13 | 11.65 ± 0.34 |
| | 8 | 104 | 11.75 ± 2.54 | 11.75 ± 3.10 |
| Day 6 | 0 | 120 | 13.27 ± 0.38 | 13.47 ± 1.42 |
| | 8 | 128 | 13.42 ± 0.49 | 13 ± 0.25 |
| Day 7 | 0 | 144 | 12.3 ± 0.63 | 12.3 ± 2.18 |
| | 8 | 152 | 12.62 ± 1.10 | 11.7 ± 1.33 |
| Day 8 | 0 | 168 | 12.72 ± 0.72 | 11.52 ± 1.07 |
| Day 9 | 0 | 192 | 14.45 ± 1.52 | 12.7 ± 0.28 |
| Day 10 | 0 | 216 | 10.38 ± 9.03 | 5.47 ± 1.13 |

All the compositions (F-1, F-2, F-3 and F-4) showed a less initial ‘burst’ and a sustained release of anastrozole for a prolonged duration. The mean plasma concentrations of 10.38±9.03 ng/ml (for F-1) and 5.47±1.13 ng/ml (for F-2) respectively were observed in last sampling point i.e. at 10 days. The mean plasma concentrations and AUC_{0-60days} of 4.24±3.55 ng/ml and 10583.4±5029.0 hr*ng/ml (for F-3) respectively, and 3.67±1.30 ng/ml and 9323.2±1046.1 hr*ng/ml (for F-4) respectively were observed at last sampling point i.e. 60 days. The Cmax (i.e. the maximum plasma concentration for a rabbit in a group considering all sampling intervals and entire study duration) observed for F-1, F-2, F-3 and F4 were 172.75±40.93 ng/ml, 127.75±19.69 ng/ml, 116.8±21.23 ng/ml and 84.03±4.39 ng/ml respectively. The considerable difference in Cmax of F-3 and F4 indicated that the oil composition (F4) gave a less Cmax and hence less burst as compared to cosolvent composition (F-3). It might be noted that though F-3 and F-4 use same microspheres (i.e. component-1); they differ in diluent composition (i.e. component-2). It was also observed that the compositions F1 and F2 having same microparticles (component-1) but different diluents (component-2) and containing/not containing a viscosity enhancing polymer(s), were giving different Cmax values. The F2 composition comprising oil as diluent (liquid vehicle) with insitu gelling polymer (viscosity enhancing agent) gave a less Cmax as compared to F1 composition comprising aqueous diluent (liquid vehicle) without insitu gelling polymer (viscosity enhancing agent). It was therefore concluded based on the study that incorporation of extra microparticle component (other than biodegradable polymer(s) in the microparticles) like insitu gelling component (viscosity enhancing agent) and diluent system (liquid vehicle) substantially assists in drug release modulation to achieve drug release with less burst effect and hence less fluctuations in plasma concentration profile.

In another embodiment of the present invention, the composition comprising component-1 and component-2 as described herein may additionally comprise at least one another component referred to as component-3. The said third component or any further component(s) might comprise diluting fluids of carriers/vehicles or solvents which might be necessary to dilute or stabilize the injectable composition or to facilitate the desired objective of achieving a sustained release of the active agent(s) from a depot formed in situ in any manner. In an embodiment, the present invention provides microparticles or nanoparticles of the active agent(s) consisting essentially of a matrix of a biocompatible and biodegradable polymer wherein the said microparticles or nanoparticles are reconstituted in a liquid vehicle such that they are substantially uniformly distributed; said active agent(s) being progressively and continuously released over a period of at least 1 day when the microparticles or nanoparticles are placed in an aqueous physiological environment, with a reduced or substantially absent first phase of accelerated release.

In an embodiment of the present invention, the injectable composition additionally comprises a thermogelling polymer which is useful to formulate the microparticles or nanoparticles, wherein the said thermogelling polymer may be present within or outside or partly within and partly outside the microparticles or nanoparticles. In another embodiment of the present invention, the composition forms an in-situ gel or gel-like structure or implant which is comprised of a network of cross-inked polymeric monomers wherein the network forms intra-network aggregates in aqueous environment of the bodily fluids. In yet another embodiment, the in-situ gel responds reversibly to a change in one or more in vivo conditions such as temperature, pH, and ionic conditions. Particularly, the in situ gel is able to imbibe or solubilize a large amount of therapeutic agent and deliver a substantially linear and sustained release of therapeutic agent under physiological conditions.

In an embodiment, the present invention provides a depot composition for parenteral administration comprising at least one active agent(s), a biocompatible lactic-acid based polymer; a polymer solvent that forms flowable gel with said biocompatible lactic-acid based polymer, wherein said polymer solvent is selected from the group consisting of triacetin, n-methyl-2-pyrrolidone, 2-pyrrolidone, glycerol formal, methyl acetate, benzyl benzoate, ethyl acetate, methyl ethyl ketone, dimethylformamide, dimethyl sulfoxide, tetrahydrofuran, caprolactam, decymethylsulfoxide, oleic acid, and 1-dodecylazacyclo-heptan-2-one and mixtures thereof; and an amount of an emulsifying agent dispersed in the form of a dispersed droplet phase in the flowable gel, wherein the emulsifying agent in combination with the polymer solvent renders said polymer solution thixotropic, said emulsifying agent selected from the group consisting of ethanol, isopropyl alcohol, and mixture thereof; and the active agent(s) homogenously dissolved or dispersed in the flowable gel; wherein the depot composition is adapted to release the active agent(s) for a substantially longer duration.

In an embodiment, it is an advantage of the injectable compositions of the present invention that the compositions upon reconstitution for injection are not very viscous. Often the viscosity enhancing polymer remains in substantially unhydrate form during injection facilitating easy injection using a standard gauge needle. Upon injection, the said polymer gets hydrated by bodily aqueous fluids forming a substantially thick gel at injection site and thereby creates a primary barrier for initial burst release of the active agent(s) from the biodegradable microparticles or nanoparticles and later provides a sustained release of the active agent(s) from the biodegradable microparticle or nanoparticle system, thus providing an option for modulating the drug release so as to obtain a sustained release of the active agent(s) for an extended period of time. The inventors of the present invention with intellectual expertise have carried out undue experimentation to prepare novel injectable depot compositions which are substantially devoid of so called ‘burst’ release of the active agent thus providing a sustained release of the active agent(s) for an extended period of time.

The compositions of the present invention are sufficiently stable so that a depot comprising one quantity or batch of the composition can provide continuous release of the composition to a patient or subject for up to at least about six months. The release of the active agent is over alternative periods of time, such as up to about one week, up to about two weeks, up to about three weeks, up to about one month, up to about two months, up to about three months, up to about four months, up to about five months, or up to about six months, or more

The use of a combination of two or more different implant or microparticle formulations according to the present invention enables a wide range of release profiles to be achieved by appropriate selection of polymers and/or loading of the active agent into the microparticles. This may be advantageous for the treatment of certain diseases. For example, it may be desirable to provide a high initial dose of the active agent(s), followed by a lower dose for the remainder of the treatment. This may be achieved by selecting a first implant or microparticle formulation which has a high initial release rate of the active agent(s) and a second implant or microparticle formulation which has a more constant release rate. The cumulative active agent(s) release from the two formulations thereby provides a high initial dose followed by a substantially constant release rate for the remainder of the treatment period. Alternatively, by appropriate selection of two or more different implant or microparticle formulations it is possible to provide a cumulative release of the active agent(s) which is substantially zero order (i.e. substantially constant) throughout the treatment period. The release profile of the active agent(s) from the first and second implants/microparticle formulations may be controlled by, for example, varying the lactide:glycolide ratio and/or the molecular weight of the polylactide or poly(lactide-co-glycolide) and/or the loading of the active agent(s) in the implant and/or the amount of the viscosity enhancing polymer.

In yet another embodiment of the present invention is provided process for preparation of such novel injectable compositions which comprises preparation of the active agent(s) microparticles or nanoparticles and a liquid vehicle in which the said microparticles or nanoparticles may be reconstituted prior to administration.

In a further embodiment, the process for preparation of compositions according to the present invention comprises of the following steps:
i) mixing the active agent(s) with biodegradable polymer(s) to form microparticles or nanoparticles,
ii) mixing the microparticles or nanoparticles of step (i) optionally with viscosity enhancing agent(s) and/or optionally with one or more excipient(s) to form component-1,
iii) mixing the liquid vehicle optionally with viscosity enhancing agent(s) and/or other excipients to form component-2, and
iv) mixing the component-1 and component-2 to obtain the desired composition before administration.

In a further embodiment, the process for preparation of compositions according to the present invention comprises of the following steps:
i) dissolving or dispersing the active agent(s) and biodegradable polymer(s) in a water immiscible solvent,
ii) homogenizing the solution of step (i) with an aqueous emulsifier solution, evaporating the solvent to form the microparticles or nanoparticles, washing and freeze drying the microparticles or nanoparticles,
iii) mixing the microparticles or nanoparticles of step (ii) optionally with viscosity enhancing agent(s) and/or optionally with one or more excipient(s) to form component-1,
iv) mixing the liquid vehicle optionally with viscosity enhancing agent(s) and/or other excipient(s) to form component-2, and
v) mixing the component-1 and component-2 to obtain the desired composition before administration.

In a further embodiment, the process for preparation of compositions according to the present invention comprises of the following steps:
i) dissolving the active agent and biodegradable polymer(s) in an appropriate solvent and spray drying to form microparticles or nanoparticles,
ii) freeze drying the microparticles or nanoparticles with appropriate cryoprotectants,
iii) mixing the microparticles or nanoparticles of step (ii) optionally with viscosity enhancing agent(s) to form component-1,
iv) mixing the liquid vehicle optionally with viscosity enhancing agent(s) and/or other excipient(s) to form component-2, and
v) mixing the component-1 and component-2 to obtain a suitable injectable dosage form composition before administration.

In a further embodiment, the inventors of the present invention had found that during the process of preparation of the microparticles or nanoparticles, when homogenization was done preferably using Ultra Turrax homogeniser for a particular time period such as for about 30 seconds at a specific speed such as about 15000 rpm, the microparticles obtained had better shape and properties. Further, the washing of microparticles or nanoparticles when carried out by repeated centrifugation and resuspension of the residue in fresh water to remove the solvent and emulsifier, produced very good microparticles or nanoparticles that were appreciably hard, had good shape and were substantially non-porous. It might be understood that the use of a suitable homogenizer and optimized process parameters such as pressure, number of cycles, flow rate of the feed, and the like for the preparation of emulsion shall produce microparticles having defined particle size, shape and other desirable characteristics. Homogenization was also done by vigorous stirring of both the phases by using a magnetic stirrer or over head stirrers with anchor or paddle stirring element. During emulsification stage, the variables like speed of stirring, shape and dimension of stirring element and vessel with reference to batch size would be precisely controlled to yield microparticles of desired shape and size. It is also desired that washing of the formed microparticles shall be carried out using cross flow or tangential flow filtration system (Minimate® TFF system from Pall Corporation), wherein the microparticles suspension is concentrated by filtration and diluted with fresh water repeatedly to wash the microparticles.

In an embodiment of the present invention, the process parameters employed during the preparation of biodegradable microparticles or nanoparticles is intended to achieve the production of the microparticles or nanoparticles with defined shape, size distribution and quantity of active pharmaceutical agent entrapped in polymer matrix in a substantially reproducible manner. In a preferred embodiment, the process employed in the present invention to produce the microparticles or nanoparticles as by w/o, o/w, w/o/w and o/w/o more preferably o/w emulsion is solvent evaporation technique known to the art. The different ingredients used to produce microparticles or nanoparticles are selected from the commonly used compounds.

In a further embodiment, in the o/w emulsion technique, the active agent(s) and the biodegradable polymer(s) were dissolved in water immiscible solvents considered as ‘oil phase’; the solution was homogenized with a ‘water phase’ containing an emulsifier. The resultant emulsion was stirred optionally with moderate heating optionally under applied vacuum so that the inner organic solvent was evaporated during agitation leaving behind the suspension of microparticles or nanoparticles formed due to hardening of biodegradable polymers from oil phase. Both the emulsifier and the organic solvents used were lost during the process and hence not present in final product or present within acceptable limits. In a process of the present invention, the organic solvent was removed by evaporation through agitation or warming, and the emulsifier was removed by washing with water. Further, the emulsifier enhances the stabilization of oil droplets against coalescence. Emulsifier concentration in the water phase strongly influences drug distribution within microparticles and release profiles. Further, emulsifier was added optionally to the water phase in order to keep the precipitating biodegradable polymer as fine independent dispersed particles.

In another embodiment of the present invention, the biodegradable microparticles or nanoparticles are produced by spray drying or lyophilization technique. In order to obtain the desired microparticles or nanoparticles, appropriate quantity of cryoprotectants is used in the composition to facilitate ready dispersibility of the composition in the diluent (vehicle) for reconstitution. Cryoprotectants such as lactose, trehalose, sucrose, or mannitol are preferably incorporated into the composition along with the biodegradable drug microparticulate form at the time of spray drying or lyophilization.

In an embodiment of the present invention, the microparticles are preferably spherical shaped. The mean particle size of microparticles is in the range of about 1 to about 250 microns, preferably about 2 to about 150 microns, and more preferably about 10 to about 100 microns as measured by a suitable technique known to the art, whereby administration of the microparticles to a subject can be carried out with a standard gauge needle. It was also observed that narrower the particle size distribution range, better was the redispersibility of microparticles in the liquid vehicle, and better was reproducibility of drug release pattern from the microparticles. In an embodiment, the injectable composition of the present invention is in the form of nanoparticles comprising active agent(s) preferably having a mean particle size range of about 1000 nm to about 2000 nm, wherein said nanoparticles are suspended in a vehicle and targeted for delivery to specific site of disease to provide a sustained release of active agent(s) for an extended time period.

In an embodiment, the composition of the present invention can be administered to a subject, animals or humans, preferably via intramuscular, intradermal, cutaneous or subcutaneous routes. Specifically the parenteral composition according to the invention can be given by any of the following routes such as among others: intra-abdominal, intra-articular, intra-capsular, intra-cervical, intra-cranial, intra-ductal, intra-dural, intra-lesional, intra-ocular, intra-locular, intramural, intra-operative, intra-parietal, intra-peritoneal, intra-plural, intra-pulmonary, intra-spinal, intrathoracic, intra-tracheal, intra-tympanic, intra-uterine or transdermal. In a preferred embodiment, the composition is in the form of parenteral composition, which may be administered via intramuscular or subcutaneous route.

In an embodiment, the in-situ gelling composition according to the present invention can deliver the active agent(s) directly to the target and provide short or long-term treatment by the controlled release of the active agent(s) in the target area. The application of the composition may be by any means necessary to introduce the active agent(s) in vivo into a subject such as a mammal including invasive surgery and/or application, preferentially, by injection. The parenteral route for delivering the compositions of the present invention is preferably selected from the group consisting of subcutaneous, intramuscular, intraorbital, intracapsular, intraspinal, intasternal, or the like. The depot formed in vivo is of a consistency selected from the group consisting of a viscous material, a gel or semi-solid, and combinations thereof. The rate of release of the active agent(s) from the depot might vary based on variation in one or more factors such as initial particle size, levels of gel in the formulation, the amount of active agent, levels of any additional materials in the formulation, the subject, subject metabolism, the administration site, and combinations thereof.

In an essential embodiment, the depot formed by the composition of the present invention traps the active agent microparticles or nanoparticles within the depot in a relatively short period of time such that any free microparticle or nanoparticle is substantially captured by the coagulating process before being carried away from the depot. For the purposes of this specification ‘depot’ is defined as a substance (preferably containing an active agent) that is retained in close proximity to the site of injection so that release of the active agent occurs over a prolonged period of time. In an embodiment, the depot erodes/dissolves in the in vivo environment of the subject over time and in doing so releases the active agent into the subject. A further advantage of the present invention is that leakage from the injection site is minimized or removed altogether. The gelling characteristics of the formulation bind the active agent microparticles or nanoparticles within close proximity of the injection site. This avoids back flow of formulation out through the injection point thus stopping unwanted waste of the agent and also gives a clean wound/administration area. In addition, the combination of microparticle or nanoparticle and polymeric delivery systems also increases design flexibility of the drug delivery system to allow a fit to individual needs. Such drug delivery systems have modified or improved release profiles and individual delivery system through modulating the drug dissolution rate and gel matrix erosion rate.

In yet another embodiment of the present invention is provided a method of forming a depot gel or an implant in situ, in a living body, which comprises preparing an in situ gelling formulation according to the method described herein, placing the formulation within the body and allowing the liquid vehicle to disperse or dissipate to produce a solid or gel implant. In yet another embodiment of the present invention is provided use of an in situ gelling formulation as described herein in the manufacture of a medicament for the treatment of a condition treatable by the active agent(s) in a mammal particularly a human being.

In yet another embodiment of the present invention is provided a method of using the compositions according to the present invention which comprises administering to a subject/patient in need thereof an effective amount of the said composition. In still another embodiment is provided use of the composition according to the present invention for the manufacture of a medicament for the prophylaxis, amelioration and/or treatment of a disease(s)/disorder(s).

The above-described exemplary embodiments are intended to be illustrative in all respects, rather than restrictive, of the present invention. Thus the present invention is capable of many variations in detailed implementation that can be derived from the description contained herein by a person skilled in the art. All such variations and modifications are considered to be within the scope and spirit of the present invention.

### EXAMPLES

### Example 1 (F-1)

| | |
|---|---|
| S. No. Ingredient | Quantity/unit dose |

Component-1

| 1. | Anastrozole | 30.0 | mg |
|---|---|---|---|
| 2. | Poly (lactide-co-glycolide) 75/25 | 270.0 | mg |
| 3. | Polyvinyl alcohol | 22.5 mg (lost in processing) | |
| 4. | Dichloromethane | 2.4 ml (lost in processing) | |
| 5. | Purified water | 5.4 ml (lost in processing) | |
| 6. | Mannitol | 60.0 | mg |

Component-2

| | | | |
|---|---|---|---|
| 1. | Sodium carboxymethyl cellulose | 30 | mg |
| 2. | Purified water | 1.5 | ml |

Procedure:
i) A solution was prepared by dissolving Polyvinyl alcohol in Purified water under stirring and cooling to room temperature by continuous stirring.
ii) Anastrozole and Poly (lactide-co-glycolide) 75/25 were dissolved in Dichloromethane and the clear solution was added to Polyvinyl alcohol solution under homogenization.
iii) The emulsion of step (ii) was stirred until Dichloromethane was completely evaporated leaving behind the suspension of microparticles.
iv) The microparticles of step (iii) were washed with water to remove Polyvinyl alcohol. The washing was carried out by repeated centrifugation at about 5° C. and resuspending the residue in fresh Purified water.
v) The finally obtained residue was dispersed in Mannitol solution, lyophilized to get free flowing powder of microparticles of Anastrozole entrapped in Poly (lactide-co-glycolide).
vi) Prepared microparticles were filled in suitable vial or prefilled syringe (component-1).
vii) Component-2 was prepared by mixing Sodium carboxymethyl cellulose and Purified water and filled in a vial.

### Example-2 (F-2)

| | |
|---|---|
| S. No. Ingredient | Quantity/unit dose |

Component-1

| 1. | Anastrozole | 30.0 | mg |
|---|---|---|---|
| 2. | Poly (lactide-co-glycolide) 75/25 | 270.0 | mg |
| 3. | Polyvinyl alcohol | 22.5 mg (lost in processing) | |
| 4. | Dichloromethane | 2.4 ml (lost in processing) | |
| 5. | Purified water | 5.4 ml (lost in processing) | |
| 6. | Mannitol | 60.0 | mg |
| 7. | Sodium carboxymethyl cellulose | 45.0 | mg |

Component-2

| | | | |
|---|---|---|---|
| 1. | Peanut oil | 1.425 | ml |
| 2. | Polysorbate 80 | 0.075 | ml |

Procedure:
i) A solution was prepared by dissolving Polyvinyl alcohol in Purified water under stirring and cooling to room temperature by continuous stirring.
ii) Anastrozole and Poly (lactide-co-glycolide) 75/25 were dissolved in Dichloromethane and the clear solution was added to Polyvinyl alcohol solution under homogenization.
iii) The emulsion of step (ii) was stirred until Dichloromethane was completely evaporated leaving behind the suspension of microparticles.
iv) The microparticles of step (iii) were washed with water to remove Polyvinyl alcohol.
v) The finally obtained residue was dispersed in Mannitol solution, lyophilized to get free flowing powder of microparticles of Anastrozole entrapped in Poly (lactide-co-glycolide).
vi) The prepared microparticles were blended with Sodium carboxymethyl cellulose and filled in suitable vial or prefilled syringe (component-1).
vii) Component-2 was prepared by mixing Peanut oil and Polysorbate 80 and filled in a vial.

### Example-3 (F-3)

| | |
|---|---|
| S. No. Ingredient | Quantity/unit dose |

Component-1

| 1. | Anastrozole | 30.0 | mg |
|---|---|---|---|
| 2. | Poly (lactide-co-glycolide) 75/25 | 600.0 | mg |
| 3. | Polyvinyl alcohol | 50.0 mg (lost in processing) | |
| 4. | Dichloromethane | 5.0 ml (lost in processing) | |
| 5. | Purified water | 10.0 ml (lost in processing) | |
| 6. | Mannitol | 60.0 | mg |
| 7. | Sodium carboxymethyl cellulose | 63.0 | mg |

Component-2

| | | | |
|---|---|---|---|
| 1. | Propylene glycol | 1.26 | ml |
| 2. | Glycerin | 0.63 | ml |
| 3. | Saline pH 7.4, Phosphate buffered | 0.21 | ml |

Procedure:
i) A solution was prepared by dissolving Polyvinyl alcohol in Purified water under stirring and cooling to room temperature by continuous stirring.
ii) Anastrozole and Poly (lactide-co-glycolide) 75/25 were dissolved in Dichloromethane and the clear solution was added to Polyvinyl alcohol solution under homogenization.
iii) The emulsion of step (ii) was stirred until Dichloromethane was completely evaporated leaving behind the suspension of microparticles.
iv) The microparticles of step (iii) were washed with water to remove Polyvinyl alcohol.
v) The finally obtained residue was dispersed in Mannitol solution, lyophilized to get free flowing powder of microparticles of Anastrozole entrapped in Poly (lactide-co-glycolide).
vi) The prepared microparticles were blended with Sodium carboxymethyl cellulose and filled in suitable vial or prefilled syringe (component-1).
vii) Component-2 was prepared by mixing Propylene glycol, Glycerin and Saline pH 7.4, Phosphate buffered and filled in a vial.

### Example-4 (F-4)

| | |
|---|---|
| S. No. Ingredient | Quantity/unit dose |

Component-1

| 1. | Anastrozole | 30.0 | mg |
|---|---|---|---|
| 2. | Poly (lactide-co-glycolide) 75/25 | 600.0 | mg |
| 3. | Polyvinyl alcohol | 50.0 mg (lost in processing) | |
| 4. | Dichloromethane | 5.0 ml (lost in processing) | |
| 5. | Purified water | 10.0 ml (lost in processing) | |
| 6. | Mannitol | 60.0 | mg |
| 7. | Sodium carboxymethyl cellulose | 63.0 | mg |

Component-2

| | | | |
|---|---|---|---|
| 1. | Peanut oil | 2.01 | ml |
| 2. | Polysorbate 80 | 0.09 | ml |

Procedure:
i) A solution was prepared by dissolving Polyvinyl alcohol in Purified water under stirring and cooling to room temperature by continuous stirring.
ii) Anastrozole and Poly (lactide-co-glycolide) 75/25 were dissolved in Dichloromethane and the clear solution was added to Polyvinyl alcohol solution under homogenization.
iii) The emulsion of step (ii) was stirred until Dichloromethane was completely evaporated leaving behind the suspension of microparticles.
iv) The microparticles of step (iii) were washed with water to remove Polyvinyl alcohol.
v) The finally obtained residue was dispersed in Mannitol solution, lyophilized to get free flowing powder of microparticles of Anastrozole entrapped in Poly (lactide-co-glycolide).
vi) The prepared microparticles were blended with Sodium carboxymethyl cellulose and filled in suitable vial or prefilled syringe (component-1).
vii) Component-2 was prepared by mixing Peanut oil and Polysorbate 80 and filled in a vial.

### Example 5

| | |
|---|---|
| S. No. Ingredient | Quantity/unit dose |

Component-1

| 1. | Donepezil | 100.0 | mg |
|---|---|---|---|
| 2. | Poly(e-caprolactone) | 700.0 | mg |
| 3. | Polyvinyl pyrrolidone | 240.0 mg (lost in processing) | |
| 4. | Dichloroethane | 10.0 ml (lost in processing) | |
| 5. | Water for Injection | 24.0 ml (lost in processing) | |
| 6. | Sucrose | 17.0 | mg |
| 7. | Hydroxyethyl cellulose | 45.0 | mg |

Component-2

| | | | |
|---|---|---|---|
| 1. | Propylene glycol | 1.4 | ml |
| 2. | Glycerin | 0.4 | ml |
| 3. | Ethanol | 0.2 | ml |

Procedure:
i) Polyvinyl pyrrolidone solution was prepared by dissolving Polyvinyl pyrrolidone in water for injection by continuous stirring.
ii) Donepezil and Poly (e-caprolactone) were dissolved in Dichloroethane and the clear solution was added to Polyvinyl pyrrolidone solution under homogenization.
iii) The emulsion of step (ii) was stirred until Dichloroethane was completely evaporated leaving behind the suspension of microparticles.
iv) The microparticles of step (iii) were washed with water for injection to remove Polyvinyl pyrrolidone.
v) The finally obtained residue of step (iv) was dispersed in Sucrose solution and lyophilized to get free flowing powder comprising microparticles of Donepezil.
vi) The microparticles of step (v) were blended with Hydroxyethyl cellulose and filled in suitable vial or prefilled syringe (component-1).
vii) Component-2 was prepared by mixing Propylene glycol, Ethanol and Glycerin.

### Example 6

| | |
|---|---|
| S. No. Ingredient | Quantity/unit dose |

Component-1

| 1. | Risperidone | 37.5 | mg |
|---|---|---|---|
| 2. | Poly(Ricenolic acid) | 700.0 | mg |
| 3. | Pentaerythritol | 240.0 mg (lost in processing) | |
| 4. | Dichloroethane | 10.0 ml (lost in processing) | |
| 5. | Water for Injection | 24.0 ml (lost in processing) | |
| 6. | Mannitol | 17.0 | mg |
| 7. | Sodium carboxymethyl cellulose | 45.0 | mg |

Component-2

| | | | |
|---|---|---|---|
| 1. | Propylene glycol | 1.5 | ml |
| 2. | Ethanol | 0.5 | ml |

Procedure:
i) Pentaerythritol solution was prepared by dissolving Pentaerythritol in water for injection.
ii) Risperidone and Poly (Ricenolic acid) were dissolved in Dichloroethane and the clear solution was added to Pentaerythritol solution under homogenization.
iii) The emulsion of step (ii) was stirred until Dichloroethane was evaporated leaving behind the suspension of microparticles.
iv) Microparticles of step (iii) were washed with water for injection to remove Pentaerythritol.
v) The finally obtained residue of step (iv) was dispersed in Mannitol solution and lyophilized to get free flowing powder comprising microparticles of Risperidone.
vi) The microparticles of step (v) were blended with Sodium carboxymethyl cellulose and filled in suitable vial or prefilled syringe (component-1).
vii) Component-2 was prepared by mixing Propylene glycol and Ethanol and filled in a vial.

### Example 7

| | |
|---|---|
| S. No. Ingredient | Quantity/unit dose |

Component-1

| 1. | Ziprasidone | 50.0 | mg |
|---|---|---|---|
| 2. | Poly(lactide-co-glycolide) | 300.0 | mg |
| 3. | Polyvinyl pyrrolidone | 200.0 mg (lost in processing) | |
| 4. | Dichloromethane | 40.0 ml (lost in processing) | |
| 5. | Water for Injection | 100.0 ml (lost in processing) | |
| 6. | Mannitol | 17.0 | mg |
| 7. | Methyl cellulose | 30.0 | mg |

Component 2

| | | | |
|---|---|---|---|
| 1. | Propylene glycol | 1.5 | ml |
| 2. | Glycerin | 0.5 | ml |

Procedure:
i) Polyvinyl pyrrolidone solution was prepared by dissolving Polyvinyl pyrrolidone in water for injection by continuous stirring.
ii) Ziprasidone and Poly(lactide-co-glycolide) were dissolved in Dichloromethane and the clear solution was added to Polyvinyl pyrrolidone solution under homogenization.
iii) The emulsion of step (ii) was stirred until Dichloromethane was completely evaporated leaving behind the suspension of microparticles.
iv) The microparticles of step (iii) were washed with water for injection to remove Polyvinyl pyrrolidone.
v) The finally obtained residue of step (iv) was dispersed in Mannitol solution and lyophilized to get free flowing powder comprising microparticles of Ziprasidone.
vi) The microparticles of step (v) were blended with Methyl cellulose and filled in suitable vial or prefilled syringe (component-1).
vii) Component-2 was prepared by mixing Propylene glycol and Glycerin and filled in a vial.

### Example 8

| | |
|---|---|
| S. No. Ingredient | Quantity/unit dose |

Component-1

| 1. | Aripiprazole | 100.0 | mg |
|---|---|---|---|
| 2. | Poly(lactide-co-glycolide) | 600.0 | mg |
| 3. | Polyvinyl alcohol | 200.0 mg (lost in processing) | |
| 4. | Dichloromethane | 40.0 ml (lost in processing) | |
| 5. | Water for Injection | 100.0 ml (lost in processing) | |
| 6. | Methyl cellulose | 50.0 | mg |

Component-2

| | | | |
|---|---|---|---|
| 1. | Propylene glycol | 1.7 | ml |
| 2. | Glycerin | 0.3 | ml |

Procedure:
i) Polyvinyl alcohol solution was prepared by dissolving Polyvinyl alcohol in water for injection under continuous stirring.
ii) Aripiprazole and Poly(lactide-co-glycolide) were dissolved in Dichloromethane and the clear solution was added to Polyvinyl alcohol solution under homogenization.
iii) The emulsion of step (ii) was stirred until Dichloromethane was completely evaporated leaving behind the suspension of microparticles.
iv) The microparticles of step (iii) were washed with water for injection to remove Polyvinyl alcohol. The washing was carried out by repeated centrifugation at 2500 rpm for 5 minutes at 5° C. and resuspending the residue in fresh Water for injection.
v) The finally obtained residue of step (iv) was lyophilized to get free flowing powder of microparticles of Aripiprazole entrapped in Poly(lactide-co-glycolide).
vi) The microparticles of step (v) were blended with Methyl cellulose and filled in suitable vial or prefilled syringe (component-1).
vii) Component-2 was prepared by mixing Propylene glycol and Glycerin and filled in a vial.

1-32. (canceled)

33. A novel injectable composition exhibiting minimal burst release comprising at least one active agent(s) or its pharmaceutically acceptable salts, derivatives, isomers, polymorphs, solvates, hydrates, analogues, enantiomers, tautomeric forms or mixtures thereof, at least one biodegradable polymer(s), at least one viscosity enhancing agent(s) and optionally one or more pharmaceutically acceptable excipient(s), wherein the compositions are formulated as reconstitutable biodegradable microparticles or nanoparticles, and wherein the said compositions are in the form of a multi-component system preferably comprising at least two components, and wherein the said compositions form an in situ gelling depot or an implant upon administration in vivo upon contact with body fluids therefore providing a prolonged release of the active agent(s) for extended periods of time.

34. A composition according to claim 33, exhibiting minimal burst of the active agent which is achieved by the formation of a substantially cohesive gel-like mass due to gradual swelling of viscosity enhancing agent(s) in the aqueous physiological-type environment sufficient to form a solid or semisolid depot gel or implant shortly after the composition is administered into a living host.

35. A composition according to claim 33, comprising at least one active agent(s) or its pharmaceutically acceptable salts, derivatives, isomers, polymorphs, solvates, hydrates, analogues, enantiomers, tautomeric forms or mixtures thereof from about 0.1% w/w to about 95% w/w, at least one biodegradable polymer(s) in an amount of from about 0.1% w/w to about 95% w/w, at least one viscosity enhancing agent(s) in an amount of from about 0. 1% w/w to about 95% w/w and optionally one or more pharmaceutically acceptable excipient(s) in an amount of from about 0. 1% to about 99.8% w/w based upon the total weight of the formulation, wherein the compositions are formulated as reconstitutable biodegradable microparticles or nanoparticles, and wherein the biodegradable polymer(s) is a polylactide polymer or a polyglycolide polymer or a poly(lactide-co-glycolide) co-polymer having an average molecular weight of from about 1,000 daltons to about 200,000 daltons, and wherein the said compositions provide a prolonged release of the active agent(s) for extended periods of time.

36. A composition according to claim 33, wherein the active agent is selected from a group comprising anastrozole, donepezil, aripiprazole, olanzapine, risperidone and ziprasidone.

37. A composition according to claim 33, wherein the mean particle size of microparticles is in the range of about 1 to about 250 microns and the mean particle size of the nanoparticles is in the range of about 1000 nm to about 2000 nm.

38. A composition according to claim 33, wherein the composition is a multi-component system comprising at least two components, component-1 and component-2.

39. A novel injectable depot composition according to claim 38, comprising of two components, wherein component-1 is in the form of a readily dispersible composition preferably microparticles or nanoparticles comprising at least one active agent(s) or its pharmaceutically acceptable salts, derivatives, isomers, polymorphs, solvates, hydrates, analogues, enantiomers, tautomeric forms or mixtures thereof and at least one biodegradable polymer(s), optionally with one or more pharmaceutically acceptable excipient(s); and wherein component-2 is in the form of a liquid vehicle for reconstitution of component-1 comprising at least one water miscible or water immiscible solvent, optionally with one or more pharmaceutically acceptable excipient(s); and wherein the compositions comprise at least one viscosity enhancing agent(s) either present in component-1 or component-2 or both.

40. A composition according to claim 39, wherein the viscosity enhancing agent(s) is present in an unhydrated form.

41. A composition according to claim 39, wherein the biodegradable microparticles or nanoparticles are partially or entirely embedded in the viscosity enhancing agent(s) which acts as release modifier upon contact with body fluids by getting hydrated and forming a gel around the biodegradable microparticles.

42. A composition according to claim 33, wherein the biodegradable polymer is selected from a group comprising lactic acid-based polymers; glycolic acid-based polymers; poly(D,L-lactide-co-glycolide); polycaprolactones; polyanhydrides; poly(sebacic acid); poly(ricenolic acid); poly(fumaric acid); poly(fatty acid dimmer); poly(terephthalic acid); poly(isophthalic acid); poly(p-{carboxyphenoxy}methane); poly(p-{carboxyphenoxy}propane); poly(p-{carboxyphenoxy}hexane); polyamines; polyurethanes; polyesteramides; polyorthoesters; polydioxanones; polyhydroxybutyrates; polyalkyene oxalates; polyamides; polyesteramides; polyurethanes; polyacetals; polyketals; polycarbonates; polyorthocarbonates; polysiloxanes; polyphosphazenes; succinates; hyaluronic acid; poly(malic acid); poly(amino acids); polyhydroxyvalerates; polyalkylene succinates; polyvinylpyrrolidone; polystyrene; synthetic celluloses; polyacrylic acids; polybutyric acid; polyvaleric acid; polyethylene glycol; polyhydroxycellulose; chitin; chitosan; polyorthoesters and copolymers, terpolymers; dimethyl isosorbide; lipids such as cholesterol, lecithin; poly(glutamic acid-co-ethyl glutamate) and the like, or mixtures thereof.

43. A composition according to claim 42, wherein the lactic acid-based polymer is polylactide or poly(D,L-lactide-co-glycolide).

44. A composition according to claim 43, wherein the poly(D,L-lactide-co-glycolide) polymer has a monomer ratio of lactic acid to glycolic acid in the range of 100:0 to about 10:90 and an average molecular weight of from about 1,000 to 200,000 daltons.

45. A composition according to claim 39, wherein the component-1 additionally comprises excipients selected from a group comprising channel forming agents, oily components, emulsifiers, preservatives, antioxidants, stabilizers or mixtures thereof.

46. A composition according to claim 45, wherein the emulsifier is selected from a group comprising polyoxyethylene sorbitan fatty acid esters; sorbitan fatty acid esters; polysorbates, polyvinyl alcohol, polyvinyl pyrrolidone, gelatin, lecithin, polyoxyethylene castor oil derivatives; tocopherol; tocopheryl polyethylene glycol succinate; tocopherol palmitate and tocopherol acetate; polyoxyethylene-polyoxypropylene co-polymers, or mixtures thereof.

47. A composition according to claim 45, wherein the channel forming agent is selected from a group comprising polyglycols, ethyl vinyl alcohols, glycerin, pentaerythritol, polyvinyl alcohols, polyvinyl pyrrolidone, vinyl pyrrolidone, N-methyl pyrrolidone, polysaccharides, saccharides, sugar alcohols, or mixtures thereof.

48. A composition according to claim 33, wherein the viscosity enhancing agent is selected from a group comprising cellulose derivatives, such as hydroxypropyl cellulose, hydroxyethyl cellulose, hydroxypropyl methyl cellulose, methylcellulose, sodium carboxymethyl cellulose and its derivatives, vinyl polymers, polyoxyethylene-polyoxypropylene polymers or co-polymers (Pluronics®), polysaccharides such as glycosaminoglycans, agar, pectin, alginic acid, dextran, starch and chitosan, proteins, poly(ethyleneoxide), acrylamide polymers, polyhydroxy acids, polyanhydrides, polyorthoesters, polyamides, polycarbonates, polyalkylenes, polyalkylene glycols, polyalkylene oxides, polyalkylene terepthalates, polyvinyl alcohols such as polyacrylic acid, polymethacrylic acid, polyvinyl pyrrolidone and polyvinyl alcohol, polyvinyl ethers, polyvinyl esters, polyvinyl halides, polyvinylpyrrolidone, polysiloxanes, polyvinyl acetates, polystyrene, polyurethanes, synthetic celluloses, polyacrylic acids, polybutyric acid, polyvaleric acid, poly(lactide-co-caprolactone), and copolymers, derivatives, and the like; or mixtures thereof.

49. A composition according to claim 48, wherein the viscosity enhancing agent is sodium carboxymethyl cellulose or methyl cellulose.

50. A composition according to claim 39, wherein the liquid vehicle (of component-2) is in the form of an aqueous vehicle comprising water and optionally water miscible solvent selected from a group comprising a water-miscible alcohol; dimethylsulfoxide; dimethylformamide; a water-miscible ether; a water-miscible nitrile; a water-miscible ketone; an amide; propylene glycol; glycerin; polyethylene glycol 400; glycofurol (tetraglycol); or mixtures thereof.

51. A composition according to claim 50, wherein the water miscible solvent is selected from a group comprising glycerin, ethanol, propylene glycol and polyethylene glycols, or mixtures thereof.

52. A composition according to claim 39, wherein the liquid vehicle is an oily vehicle comprising at least one oily component selected from a group comprising vegetable oils such as corn oil, almond oil, sunflower oil, peanut oil, olive oil, castor oil, soybean oil, safflower oil, cottonseed oil, and the like, or a lipophilic compound such as dimethyl isosorbide.

53. A composition according to claim 39, wherein the component-2 additionally comprises one or more of co-surfactants, co-solvents, hydrophilic solvents, preservatives, antioxidants, anti-foaming agents, stabilizers, buffering agents, pH adjusting agents, osmotic agents, isotonicity producing agents, or mixtures thereof.

54. A composition according to claim 33, wherein the composition additionally comprises a thermogelling or hydrogelling polymer.

55. A composition according to claim 33, which can be administered to a subject through the intramuscular, intradermal, cutaneous or subcutaneous, intra-abdominal, intra-articular, intra-capsular, intra-cervical, intra-cranial, intra-ductal, intra-dural, intra-lesional, intra-ocular, intra-locular, intra-mural, intra-operative, intra-parietal, intra-peritoneal, intra-plural, intra-pulmonary, intra-spinal, intrathoracic, intra-tracheal, intra-tympanic, intra-uterine or transdermal route.

56. A process for the preparation of injectable composition according to claim 33, which comprises preparation of microparticles or nanoparticles and a liquid vehicle in which the said microparticles or nanoparticles may be reconstituted prior to administration.

57. A process for the preparation of injectable composition according to claim 33, which comprises of the following steps:
i) mixing the active agent(s) with biodegradable polymer(s) to form microparticles or nanoparticles,
ii) mixing the microparticles or nanoparticles of step (i) optionally with viscosity enhancing agent(s) and/or optionally with one or more excipient(s) to form component-1,
iii) mixing the liquid vehicle optionally with viscosity enhancing agent(s) and/or other excipients to form component-2, and
iv) mixing the component-1 and component-2 to obtain the desired composition before administration.

58. A process for the preparation of injectable composition according to claim 33, which comprises of the following steps:
i) dissolving or dispersing the active agent(s) and biodegradable polymer(s) in a water immiscible solvent,
ii) homogenizing the solution of step (i) with an aqueous emulsifier solution, evaporating the solvent to form the microparticles or nanoparticles, washing and freeze drying the microparticles or nanoparticles,
iii) mixing the microparticles or nanoparticles of step (ii) optionally with viscosity enhancing agent(s) and/or optionally with one or more excipient(s) to form component-1,
iv) mixing the liquid vehicle optionally with viscosity enhancing agent(s) and/or other excipient(s) to form component-2, and
v) mixing the component-1 and component-2 to obtain the desired composition before administration.

59. A process for the preparation of injectable composition according to claim 33, which comprises of the following steps:
i) dissolving the active agent and biodegradable polymer(s) in an appropriate solvent and spray drying to form microparticles or nanoparticles,
ii) freeze drying the microparticles or nanoparticles with appropriate cryoprotectants,
iii) mixing the microparticles or nanoparticles of step (ii) optionally with viscosity enhancing agent(s) to form component-1,
iv) mixing the liquid vehicle optionally with viscosity enhancing agent(s) and/or other excipient(s) to form component-2, and
v) mixing the component-1 and component-2 to obtain a suitable injectable dosage form composition before administration.

60. A method of forming a depot gel or an implant in situ, in a living body, which comprises preparing an in situ gelling formulation according to claim 33, placing the formulation within the body and allowing the liquid vehicle to disperse or dissipate to produce a solid or gel implant.

61. A pharmaceutical kit suitable for in situ formation of a biodegradable depot gel or implant from the novel compositions according to claim 33, in the body of a subject in need thereof, which comprises a device containing microparticles comprising at least one active agent(s) and optionally one or more pharmaceutical acceptable excipient(s), and a device containing liquid vehicle and optionally one or more pharmaceutical acceptable excipient(s); wherein the devices allow for expulsion of the contents of the two devices for enabling mixing together prior to administration of the contents into the body of the subject.

62. A method of using the compositions according to claim 33, for the treatment of chronic diseases/disorders such as cancers, psychosis; amyotrophic lateral sclerosis; cerebral ischemia; Paget's disease; unstable angina and disorders due to abnormal cell proliferation.
